(19) United States
(12) Patent Application Publication
(10) Pub. No.: US 2004/0054171 A1
(43) Pub. Date: Mar. 18, 2004

(54) Polymorphic forms of a 4H-thieno[3,2-E]-1,2,4-thiadiazine 1,1-dioxide derivative

(76) Inventors: Anette Frost Jensen, Copenhagen O (DK); Finn Broni Junager, Charlottenlund (DK); Claus Ulrich Jessen, Vanloese (DK); Hanne T efting Kornoe, Lyngby (DK); Peter Palitzsch, Radebeul (DE)

Correspondence Address: Reza Green, Esq., Novo Nordisk Pharmaceuticals, Inc., 100 College Road West, Princeton, NJ, 08540, US

(21) Appl. No.: 10/608,328

(22) Filed: Jun. 27, 2003

Related U.S. Application Data
(60) Provisional application No. 60/396,045, filed on Jul. 10, 2002.

(30) Foreign Application Priority Data
Jul. 4, 2002 (DK) PA 2002 01047

(51) Int. Cl. C07D 491/02

(52) U.S. Cl. 544/10

(57) ABSTRACT

The present invention relates to novel polymorphic/pseudopolymorphic forms of 6-Chloro-3-(1-methylcyclopropyl)amino-4H-thieno[3,2-e]-1,2,4-thiadiazine 1,1-dioxide or mixtures thereof, their preparation and their use as therapeutic agents.

### CROSS REFERENCE TO RELATED APPLICATIONS

This application claims priority from U.S. Provisional application No. 60/396045, filed Jul. 9, 2002 and Danish application number PA 2002 01047 filed Jul. 4, 2002, the contents of both of which are incorporated by reference herein in their entirety.

### FIELD OF THE INVENTION

The present invention relates to novel polymorphic/pseudopolymorphic forms of the potassium channel opener 6-chloro-3-(1 -methylcyclopropyl)amino-4H-thieno[3,2-e]-1,2,4-thiadiazine 1,1-dioxide, their preparations, pharmaceutical compositions comprising the novel forms and their use as therapeutic agents.

### BACKGROUND OF THE INVENTION

Potassium channels play an important role in the physiological and pharmacological control of cellular membrane potential. Amongst the different types of potassium channels are the ATP-sensitive (K_{ATP}-) channels, which are regulated by changes in the intracellular concentration of adenosine triphosphate. The K_{ATP}-channels have been found in cells from various tissues such as cardiac cells, pancreatic cells, skeletal muscles, smooth muscles, central neurons and adenohypophysis cells. The channels have been associated with diverse cellular functions for example hormone secretion (insulin from pancreatic beta-cells, growth hormone and prolactin from adenohypophysis cells), vasodilation (in smooth muscle cells), cardiac action potential duration, neurotransmitter release in the central nervous system.

Modulators of the K_{ATP}-channels have been found to be of importance for the treatment of various diseases. Certain sulphonylureas, which have been used for the treatment of non-insulin-dependent diabetes mellitus, act by stimulating insulin release through an inhibition of the K_{ATP}-channels on pancreatic beta-cells.

The potassium channel openers, which comprise a heterogeneous group of compounds, have been found to be able to relax vascular smooth muscles and have therefore been used for the treatment of hypertension.

In addition, potassium channel openers can be used as bronchodilators in the treatment of asthma and various other diseases. Furthermore, potassium channel openers have been shown to promote hair growth, and have been used for the treatment of baldness. Potassium channel openers are also able to relax urinary bladder smooth muscle and therefore, can be used for the treatment of urinary incontinence. Potassium channel openers, which relax smooth muscle of the uterus, can be used for treatment of premature labour. Further, potassium channel openers are found to be useful in the treatment of benign prostatic hyperplasia, erectile dysfunction and in contraception.

Potassium channel openers, which inhibit insulin secretion by activating potassium channels of the beta-cell can be used alone or in combination with other compounds in order to treat or prevent Type-2 diabetes (non-insulin dependent diabetes mellitus, NIDDM), to prevent or intervent Type-1 diabetes (insulih dependent diabetes mellitus, IDDM) including prevention or slowing of progression of impaired fasting glucose (IFG) and impaired glucose tolerance (IGT).

Since some K_{ATP}-openers are able to antagonize vasospasms in basilar or cerebral arteries they can be used for the treatment of vasospastic disorders such as subarachnoid haemorrhage and migraine.

Potassium channel openers hyperpolarize neurons and inhibit neurotransmitter release and it is expected that these compounds can be used for the treatment of various diseases of the central nervous system, e.g. epilepsia, ischemia and neurodegenerative diseases, and for the management of pain.

WO 00/37474 discloses a class of potassium channel openers having the general formula

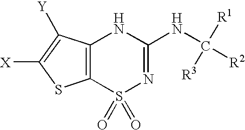

wherein X and Y independently are hydrogen, halogen, perhalomethyl, C₁₋₆-alkyl or C₁₋₆-alkoxy; R¹, R² and R³ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polyubstituted with halogen, hydroxy, oxo, or aryl; or R¹ is as defined above and R²—C—R³ form a C₃₋₆-cycloalkyl group, optionally being mono- or polysubstituted with C₁₋₆-alkyl, perhalomethyl, halogen, hydroxy or aryl; or —CR¹R²R³ form a 4- to 12-membered bicyclic or tricyclic carbocyclic system, optionally being mono- or polysubstituted with C₁₋₆-alkyl, perhalomethyl, halogen, hydroxy or aryl; or a salt thereof with a pharmaceutically acceptable acid or base including all optical isomers of compounds of the formula , some of which are optically active, and also their mixtures including racemic mixtures, or any tautomeric form thereof. Particularly the compounds disclosed include 6-chloro-3-(1-methylcyclopropyl)amino-4H-thieno[3,2-e]-1,2,4-thiadiazine 1,1-dioxide.

The crystalline behaviour of such compounds and particularly that of 6-chloro-3-(1-methylcyclopropyl)amino-4H-thieno[3,2-e]-1,2,4-thiadiazine 1,1-dioxide has been investigated in order to explore the existence of other polymorphic forms of the compound and to establish thermodynamically stable/thermodynamically metastable polymorphs.

Thermodynamically stable polymorphs and polymorphs, which have shown to be thermodynamically metastable over a considerable period of time, may have advantages in higher solubility. Thus, thermodynamically stable/thermodynamically metastable polymorphs may possess higher bioavailability in human beings. Further, different polymorphic and pseudopolymorphic forms may also differ in hygroscopicity, possess different other physical properties such as tap and bulk density, angle of rest, particle size, or may possess different stabilities at various storage conditions, all of which are important parameters during formulation.

It has now been found that various novel polymorphic/pseudopolymorphic crystal forms of 6-chloro-3-(1-methylcyclopropyl)amino-4H-thieno[3,2-e]-1,2,4-thiadiazine 1,1-dioxide of formula (I)

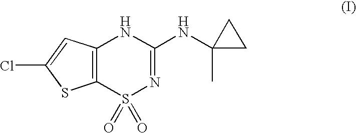

have advantageous physico-chemical characteristics that will ease the handling of the compound. In addition, the novel crystalline polymorphs designated A, B and D are pharmacologically advantageous to the crystal form of 6-chloro-3-(1-methylcyclopropyl)amino-4H-thieno[3,2-e]-1,2,4-thiadiazine 1,1-dioxide disclosed in WO 00/37474, Example 3, since they are solvent free and thus posses no toxic potential from this source.

### DESCRIPTION OF THE DRAWINGS

The present invention is further described in the appended drawings in which:

FIG. 1 illustrates the DSC thermogram of polymorph A

FIG. 2 illustrates the X-ray powder diffractogram of polymorph A

FIG. 3 illustrates the DSC thermogram of polymorph B

FIG. 4 illustrates the X-ray powder diffractogram of polymorph B

FIG. 5 illustrates the DSC thermogram of pseudopolymorph C1, a solvate of acetone

FIG. 6 illustrates the TGA trace of pseudopolymorph C1, a solvate of acetone

FIG. 7 illustrates the X-ray powder diffractogram of pseudopolymorph C1, a solvate of acetone

FIG. 8 illustrates the DSC thermogram of pseudopolymorph C2, a solvate of 1-butanol

FIG. 9 illustrates the TGA trace of pseudopolymorph C2, a solvate of 1-butanol

FIG. 10 illustrates the X-ray powder diffractogram of pseudopolymorph C2, a solvate of 1-butanol

FIG. 11 illustrates the DSC thermogram of pseudopolymorph C3, a solvate of 2-butanol

FIG. 12 illustrates the TGA trace of pseudopolymorph C3, a solvate of 2-butanol

FIG. 13 illustrates the X-ray powder diffractogram of pseudopolymorph C3, a solvate of 2-butanol

FIG. 14 illustrates the DSC thermogram of pseudopolymorph C4, a solvate of 1,4-dioxane

FIG. 15 illustrates the TGA trace of pseudopolymorph C4, a solvate of 1,4-dioxane

FIG. 16 illustrates the X-ray powder diffractogram of pseudopolymorph C4, a solvate of 1,4-dioxane

FIG. 17 illustrates the DSC thermogram of pseudopolymorph C5, a solvate of methylacetate

FIG. 18 illustrates the TGA trace of pseudopolymorph C5, a solvate of methylacetate

FIG. 19 illustrates the X-ray powder diffractogram of pseudopolymorph C5, a solvate of methylacetate

FIG. 20 illustrates the DSC thermogram of pseudopolymorph C6, a solvate of methylethylketone

FIG. 21 illustrates the TGA trace of pseudopolymorph C6, a solvate of methylethylketone

FIG. 22 illustrates the X-ray powder diffractogram of pseudopolymorph C6, a solvate of methylethylketone

FIG. 23 illustrates the DSC thermogram of pseudopolymorph C7, a solvate of THF

FIG. 24 illustrates the TGA trace of pseudopolymorph C7, a solvate of THF

FIG. 25 illustrates the X-ray powder diffractogram of pseudopolymorph C7, a solvate of THF

FIG. 26 illustrates the DSC thermogram of pseudopolymorph C8, a solvate of toluene

FIG. 27 illustrates the TGA trace of pseudopolymorph C8, a solvate of toluene

FIG. 28 illustrates the X-ray powder diffractogram of pseudopolymorph C8, a solvate of toluene

FIG. 29 illustrates the DSC thermogram of pseudopolymorph C9, a solvate of 1-pentanol

FIG. 30 illustrates the TGA trace of pseudopolymorph C9, a solvate of 1-pentanol

FIG. 31 illustrates the X-ray powder diffractogram of pseudopolymorph C9, a solvate of 1-pentanol

FIG. 32 illustrate the DSC thermogram of polymorph D

FIG. 33 illustrates the X-ray powder diffractogram of polymorph D

FIG. 34 illustrates the X-ray powder diffractogram of a mixture of polymorphs A and B

FIG. 35 illustrates the X-ray powder diffractogram of a mixture of polymorph B and pseudopolymorph Cⱼ

### DESCRIPTION OF THE INVENTION

The present invention relates to novel polymorphic/pseudopolymorphic forms or mixtures thereof of the potassium channel opener 6-chloro-3-(1-methylcyclopropyl)amino-4H-thieno[3,2-e]-1,2,4-thiadiazine 1,1 -dioxide of formula (I)

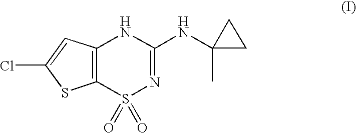

and pharmaceutically acceptable solvates thereof and pharmaceutical compositions containing them or their mixtures.

A number of polymorphic/pseudopolymorphic forms of 6-chlor-6-3- (1-methylcyclopropyl)amino-4H-thieno[3,2-e]-1,2,4-thiadiazine 1,1-dioxide or mixtures thereof have been identified. The polymorphic forms identified have been designated A, B and D. The pseudopolymorphic forms identified are all isostructural, i.e. they possess the same crystal structure and have therefore been designated Cj, j=1, 2, 3, 4, 5, 6, 7, 8 and 9. The mixtures of polymorphic/pseudopolymorphic forms identified have been designated AB and BC. The polymorphic/pseudopolymorphic forms or mixtures thereof may be obtained from the following solvents: acetic acid, acetone, anisole, 1-butanol, 2-butanol, butylacetate, butylmethylether, cumene, DMSO (dimethylsulfoxide), ethanol, 1-propanol, 2-propanol, ethylacetate, ethylether, ethylformate, formic acid, heptane, iso-butylacetate, iso-propylacetate, methanol, methylacetate, 3-methyl-1-butanol, methylethyl ketone, methyl iso-butyl ketone, 2-methyl-1-propanol, pentane, 1-pentanol, propanolacetate, water or any combinations thereof.

Within another aspect of the invention there is provided a pharmaceutical composition comprising a polymorphic/pseudopolymorphic form of 6-chloro-3-(1-methylcyclopropyl)amino-4H-thieno[3,2-e]-1,2,4-thiadiazine 1,1-dioxide or a mixture thereof, optionally in combination with one or more pharmaceutically acceptable carriers or excipients.

Within another aspect of the invention there is provided a process for the preparation of the polymorphic/pseudopolymorphic forms of 6-chloro-3-(1-methylcyclopropyl)amino-4H-thieno[3,2-e]-1,2,4-thiadiazine 1,1-dioxide or mixtures thereof, which processes comprises suspending or dissolving 6-chloro-3-(1-methylcyclopropyl)amino-4H-thieno[3,2-e]-1,2,4-thiadiazine 1,1-dioxide in an appropriate solvent or a mixtures of solvents and crystallizing the various forms from the solution.

The process for the preparation of the various polymorphic forms of the present invention comprises:

a) suspending or dissolving 6-chloro-3-(1-methylcyclopropyl)amino-4H-thieno[3,2-e]-1,2,4-thiadiazine 1,1-dioxide in an appropriate solvent or a mixture of solvents,

b) optionally heating the mixture to 60-120° C. depending on the boiling point of the appropriate solvent or solvent mixture so that the solution becomes clear, and filtering the clear solution,

c) optionally adding a co solvent at 60-120° C.,

d) optionally distilling off solvent,

e) slowly cooling the solution to 0-50° C., or adding the solution to a third solvent or mixture of solvents, or adding solvent or a mixture of solvents to the solution or combinations thereof whereby crystals are formed,

g) filtrating the resulting suspension,

h) washing the filter cake with an appropriate solvent or mixture of solvents and drying the filter cake to constant weight.

The various processes will be described in detail in the following examples.

Examples of solvents include but are not limited to: water, hydrocarbons (aromatic, aliphatic, unsaturated, aromatic) such as pentane, heptane, cumene or toluene; alcohols (monohydric or polyhydric aliphatic, unsaturated, aromatic) such as methanol, ethanol, 1-propanol, 2-propanol, 2-methyl-1-propanol, 1-butanol, 2-butanol, 1-pentanol; ethers (open chain or cyclic) such as ethyl ether, tert-butyl methyl ether, anisole, 1,4-dioxane or tetrahydrofurane; carbonyls (aldehydes, ketones) such as acetone, methyl ethyl ketone, methyl isobutyl ketone; carbonic acids such as formic acid, acetic acid; carbonic acid esters (mono or poly saturated aliphatic, unsaturated or aromatic) such as ethyl formiate, methyl acetate, ethyl acetate, propyl acetate, isopropyl acetate, n-butyl acetate, isobutyl acetate, tert butyl acetate; carbonates such as dimethylcarbonate; halogenated hydrocarbons such as dichloromethane; solvents containing nitrogen (nitriles, amines, nitro, amides ureas) such as acetonitril, N,N-dimethylformamide, 1-methyl-2-pyrrolidinone, nitromethane, 1,3-dimethyl-3,4,5,6-tetrahydroxy-2(1H)-pyrimidinone; oxosulfor compounds such as sulfolane, dimethylsulfoxide, or combinations thereof.

In a further aspect, the present invention relates to the use of the polymorphic/pseudopolymorphic forms of 6-chloro-3-(1-methylcyclopropyl) amino-4H-thieno[3,2-e]-1,2,4-thiadiazine 1,1-dioxide or mixtures thereof, for the preparation of a pharmaceutical composition for the treatment of various diseases of the cardiovascular system, e.g. cerebral ischemia, hypertension, ischemic heart diseases, angina pectoris and coronary heart diseases; the pulmonary system; the gastrointestinal system; the central nervous system and the endocrinological system.

In a further aspect, potassium channel openers hyperpolarize neurons and inhibit neurotransmitter release and hence the polymorphic/pseudopolymorphic forms of 6-chloro-3-(1-methylcyclopropyl)amino-4H-thieno[3,2-e]-1,2,4-thiadiazine 1,1-dioxide or mixtures thereof, can be used in the preparation of a pharmaceutical composition for the treatment of various diseases of the central nervous system, e.g. epilepsia, ischemia and neurodegenerative diseases, and for the management of pain.

In a further aspect, potassium channel openers promote hairgrowth, therefore, the polymorphic/pseudopolymorphic forms of 6-chloro-3-(1-methylcyclopropyl)amino-4H-thieno[3,2-e]-1,2,4-thiadiazine 1,1-dioxide or mixtures thereof, can be used for the for the preparation of a pharmaceutical composition for the treatment of baldness.

In diseases such as nesidioblastosis and insulinoma in which a hypersecretion of insulin causes severe hypoglycemia the polymorphic/pseudopolymorphic forms of 6-chloro-3-(1-methylcyclopropyl)amino-4H-thieno[3,2-e]-1,2,4-thiadiazine 1,1-dioxide or mixtures thereof, can be used to reduce insulin secretion. In obesity hyperinsulinemia and insulin resistance is very frequently encountered. This condition could lead to the development of non-insulin dependent diabetes (NIDDM). Potassium channel openers, and hence the polymorphic/pseudopolymorphic forms of 6-chloro-3-(1-methylcyclopropyl)amino-4H-thieno[3,2-e]-1,2,4-thiadiazine 1,1-dioxide or mixtures thereof, can be used for counteracting the hyperinsulinemia and thereby prevent diabetes and reduce obesity. In overt NIDDM, treatment of hyperinsulinemia with potassium channel openers, and hence the present polymorphic/pseudopolymorphic forms of 6-chloro-3-(1-methylcyclopropyl)amino-4H-thieno[3,2-e]-1,2,4-thiadiazine 1,1-dioxide or mixtures thereof, can be of benefit in restoring glucose sensitivity and normal insulin secretion. Thus, the polymorphic/pseudopolymorphic forms of the compound 6-Chloro-3-(1-methylcyclopropyl)amino-4H-thieno[3,2-e]-1,2,4-thiadiazine 1,1-dioxide or mixtures thereof, can be used for the preparation of a pharmaceutical composition for the treatment or prevention of Type-2 diabetes or Type-2 prevention in prior GDM.

In a further aspect, in early cases of insulin dependent diabetes (IDDM) or in pre-diabetic cases, potassium channel openers and hence the novel polymorphic/pseudopolymorphic forms of 6-chloro-3-(1-methylcyclopropyl)amino-4H-thieno[3,2-e]-1,2,4-thiadiazine 1,1-dioxide or mixtures thereof, can be used to induce pancreatic beta-cell rest which may prevent the progression of the autoimmune disease.

In a further aspect, the potassium channel openers of the present invention can be administred in combination with an immIunosuppressant or with an agent like nicotinamide, which will reduce autoimmune degeneration of beta-cells.

Combining beta-cell rest with a treatment protecting the beta-cells against cytokine mediated beta-cell impairment/cytotoxicity is another aspect of this invention.

Insulin requiring or Type-1 diabetes (IDDM) as well as late onset IDDM (also known as type 1.5. e.g. non-insulin-requiring Type-2 diabetes (NIDDM) patients with autoreactivity against beta-cell epitopes that later turns insulin requiring) have circulating autoreactive monocytes/lymphocytes that homes to the islets/beta-cells and releases their cytokines. Some of these cytokines (e.g. interleukin-1b (IL-1b), tumour necrosis factor a (TNFa) and interferon g (IFNg)) are specifically toxic to the beta-cells, e.g. through the induction of nitric oxide (NO) and other free radicals. Inhibition of this cytotoxicity, e.g. by co-administring nicotinamide (NA), a derivative hereof or other cytokine protective compounds to the prediabetic/diabetic patients treated with the potassium channel opener compound is an example of this aspect. Nicotinamide belongs to the B-vitamin family and is derived from nicotinic acid by amidation of the carboxyl group. It processes none of nicotine's pharmacological properties. NA is converted into NAD+, which acts as a coenzyme for proteins involved in tissue respiration. NA has been proposed to influence several of the putative intracellular molecular events following immune attack on the beta-cells. Animal experiments and early non-blinded experiments in humans have indicated a protective role of this compound against IDDM as well as in cytokine/immune mediated beta-cell destruction.

In a further aspect, the application concerns the use of the polymorphic/pseudopolymorphic forms of 6-chloro-3-(1-methylcyclopropyl)amino-4H-thieno[3,2-e]-1,2,4-thiadiazine 1,1-dioxide alone or in combination with the inhibitor of cytokine/immune mediated beta-cell impairment, in transplantation, e.g. islet transplantation into diabetes patients. The use of one or both of these treatments may reduce the risk of rejection of the transplanted islets/beta-cells/engineered beta-cells/pancreas.

In a further aspect of the invention, the polymorphic/pseudopolymorphic forms of 6-chloro-3-(1-methylcyclopropyl)amino-4H-thieno[3,2-e]-1,2,4-thiadiazine 1,1-dioxide or mixtures thereof, may be used for the preparation of a pharmaceutical composition for treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity.

In a further aspect, the present invention relates to polymorphic/pseudopolymorphic forms of 6-chloro-3-(1-methylcyclopropyl)amino-4H-thieno[3,2-e]-1,2,4-thiadiazine 1,1-dioxide or mixtures thereof, for use as a therapeutically acceptable substances, preferably for use as therapeutically acceptable substances in the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity.

In a further aspect, the invention also relates to the use of the novel polymorphic/pseudopolymorphic forms of 6-chloro-3-(1-methylcyclopropyl)amino-4H-thieno[3,2-e]-1,2,4-thiadiazine 1,1-dioxide or mixtures thereof, as pharmaceutical compositions useful for treating or preventing diseases of the endocrinological system, such as treating hyperinsulinaemia, treating or preventing Type-2 diabetes, preventing Type-2 diabetes in priorGDM, preventing and interventing Type-1 diabetes, prevention or slowing of progression of impaired fasti ng glucose (IFG) and impaired glucose tolerance (IGT), gestational diabetes mellitus (GDM) or obesity.

In this application treatment or prevention is defined as the management and care of a patient for the purpose of combating the disease, condition, or disorder and includes the administration of the active compounds to prevent the onset of the symptoms or complications, or alleviating the symptoms or complications, or eliminating the disease, condition, or disorder.

Further, in this application prevention and intervention of Type-1 diabetes is defined as follows: prevention is defined as the management and care of an individual at risk of developing Type 1 diabetes prior to the clinical onset of the disease. Intervention is defined as the management and care of a Type 1 diabetes patient at diagnosis or later. The purpose of prevention and intervention is to combat the disease, condition, or disorder and includes the administration of the active compounds to prevent or delay the onset of the symptoms or complications, or alleviating the symptoms or complications, or eliminating the disease, condition, or disorder.

In a further aspect, the pharmaceutical composition of the invention may comprise a polymorphic/pseudopolymorphic form of 6-chloro-3-(1-methylcyclopropyl)amino-4H-thieno[3,2-e]-1,2,4-thiadiazine 1,1-dioxide combined with one or more other pharmacologically active compounds, e.g. an antidiabetic or other pharmacologically active material. Suitable antidiabetics comprise short and long acting insulins, insulin analogues, insulin sensitizers, insulin secretagogues as well as orally active hypoglycaemic agents such as sulphonylureas, e.g. glibenclamide and glipizide; biguanides, e.g. metformin; benzoic acid derivatives, e.g. repaglinide; thiazolidinediones, e.g. rosiglitazone, pioglitazone and ciglitazone; glucagon like peptide 1 (GLP-1), GLP-1 derivatives and GLP-1 analogues; peroxisome proliferating activated receptor (PPAR) ligands including the PPAR-alpha, PPAR-gamma and PPAR-delta subtypes; inhibitors of α-glucosidase, e.g. acarbose and voglibose, inhibitors of hepatic enzymes responsible for the biosynthesis of glucose, e.g. glycogen phosphorylase inhibitors.

In a further aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, may be administered in combination with nateglinide.

In a further aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, may be administered in combination with one or both of metformin and repaglinide.

In still another embodiment of the invention the present compounds are administered in combination with a thiazolidinedione insulin sensitizer.eg troglitazone, ciglitazone, pioglitazone, rosiglitazone, isaglitazone, darglitazone, englitazone, CS-011/CI-1037 or T 174 or the compounds disclosed in WO 97/41097, WO 97/41119, WO 97/41120, WO 00/41121 and WO 98/45292 (Dr. Reddy's Research Foundation), which are incorporated herein by reference.

In still another embodiment of the invention the present compounds may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the present compounds are administered in combination with an α-glucosidase inhibitor eg voglibose, emiglitate, miglitol or acarbose.

In another embodiment of the invention the present compounds are administered in combination with an agent acting on the ATP-dependent potassium channel of the β-cells eg tolbutamide, glibenclamide, glipizide, glicazide, BTS-67582 or repaglinide.

In yet another embodiment of the invention the present compounds may be administered in combination with nateglinide.

In still another embodiment of the invention the present compounds are administered in combination with an antilipidemic agent eg cholestyramine, colestipol, clofibrate, gemfibrozil, lovastatin, pravastatin, simvastatin, probucol or dextrothyroxine.

In another embodiment of the invention, the present compounds are administered in combination with more than one of the above-mentioned compounds eg in combination with metformin and a sulphonylurea such as glyburide; a sulphonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In a further aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, reduces blood glucose and triglyceride levels and are accordingly useful for the treatment and/or prevention of ailments and disorders such as diabetes and/or obesity.

In another aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, are useful for the treatment and/or prophylaxis of dyslipidemia, disorders related to Syndrome X such as hypertension, obesity, insulin resistance, hyperglycaemia, atherosclerosis, hyperlipidemia, coronary artery disease, myocardial ischemia and other cardiovascular disorders.

In a further aspect, the polymorphic/pseudopolymorphic forms of 6-chloro-3-(1-methylcyclopropyl)amino-4H-thieno[3,2-e]-1,2,4-thiadiazine 1,1 dioxide or mixtures thereof, may also be useful fore treating diabetic complications, gestational diabetes mellitus (GDM), polycystic ovarian syndrome (PCOS) and smoke reduction or cessation.

Pharmaceutical compositions containing the polymorphic/pseudopolymorphic forms of 6-chloro-3-(1-methylcyclopropyl)amino-4H-thieno[3,2-e]-1,2,4-thiadiazine 1,1-dioxide or mixtures thereof, and optionally other compounds as mentioned underneath may be prepared by conventional techniques, e.g. as described in Remington: The Science and Practise of Pharmacy, 19^{th} Ed., 1995. The compositions may appear in conventional forms, for example capsules, tablets, aerosols, solutions, suspensions or topical applications.

The polymorphic/pseudopolymorphic forms of 6-chloro-3-(1-methylcyclopropyl)amino-4H-thieno[3,2e]-1,2,4-thiadiazine 1,1-dioxide or mixtures thereof, may also be administered in combination with one or more further pharmacologically active substances eg. selected from antiobesity agents, antidiabetics, antihypertensive agents, agents for the treatment and/or prevention of complications resulting from or associated with diabetes and agents for the treatment and/or prevention of complications and disorders resulting from or associated with obesity.

Thus, in a further aspect of the invention the polymorphic/pseudopolymorphic forms of 6-chloro-3-(1-methylcyclopropyl)amino-4H-thieno[3,2-e]- 1,2,4-thiadiazine 1,1-dioxide or mixtures thereof, may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds. TRH (thyreotropin releasing hormone) agonists, UCP 2 or 3 (uncoupling protein 2 or 3) modulators, leptin agonists, DA agonists (bromrocriptin, doprexin), lipase/amylase inhibitors, PPAR (peroxisome proliferator-activated receptor) modulators RXR (retinoid X receptor), modulators, TR β agonists AGRP (Agouti related protein) inhibitors, H3 histamine antagonists, opioid antagonists (such as naltrexone), exendin-4, GLP-1 and ciliary neurotrophic factor.

Suitable antidiabetics comprise insulin, GLP-1 (glucagon like peptide-1) derivatives such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as HMG CoA inhibitors (statins), and compounds lowering food intake.

In a further aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, may be administered in combination with an antihyperlipidemic agent or antilipidemic agent eg. cholestyramine, colestipol, clofibrate, gemfibrozil, lovastatin, pravastatin, simvastatin, probucol or dextrothyroxine.

In a further aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, may be administered in combination with. more than one of the above-mentioned compounds eg. in combination with a sulphonylurea and metformin, a sulphonylurea and acarbose, repaglinide and metformin, insulin and a sulphonylurea, insulin and mefformin, insulin, insulin and lovastatin, etc.

In a further aspect, the polymorphic/pseudopolymorphic forms of 6-chloro-3-(1-methylcyclopropyl)amino-4H-thieno[3,2-e]-1,2,4-thiadiazine 1,1-dioxide or mixtures thereof, may be administered in combination with one or more antihypertensive agents. Examples of antihypertensive agents are β-blockers such as alprenoiol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the polymorphic/pseudopolymorphic forms of 6-chloro-3-(1-methylcyclopropyl)amino-4H-thieno[3,2-e]-1,2,4-thiadiazine 1,1-dioxide according to the invention with one or more of the above-mentioned compounds and optionally one or more further pharmacologically active substances are considered to be within the scope of the present invention.

### PHARMACEUTICAL COMPOSITIONS

Typical compositions include the polymorphic/pseudopolymorphic forms of 6-chloro-3-(1-methylcyclopropyl)amino-4H-thieno[3,2-e]-1,2,4-thiadiazine 1,1-dioxide or mixtures thereof, associated with a pharmaceutically acceptable excipient, which may be a carrier or a diluent or be diluted by a carrier, or enclosed within a carrier, which can be in the form of a capsule, sachet, paper or other container. In making the compositions, conventional techniques for the preparation of pharmaceutical compositions may be used. For example, the active polymorphic/pseudopolymorphic forms of 6-chloro-3-(1-methylcyclopropyl)amino-4H-thieno[3,2-e]-1,2,4-thiadiazine 1,1-dioxide or mixtures thereof, will usually be mixed with a carrier, or diluted by a carrier, or enclosed within a carrier, which may be in the form of a ampoule, capsule, sachet, paper, or other container. When the carrier serves as a diluent, it may be solid, semi-solid, or liquid material, which acts as a vehicle, excipient, or medium for the active compound. The active polymorphic/pseudopolymorphic forms of 6-chloro-3-(1-methylcyclopropyl)amino-4H-thieno[3,2-e]-1,2,4-thiadiazine 1,1-dioxide or mixtures thereof, can be adsorbed on a granular solid container for example in a sachet. Some examples of suitable carriers are water, salt solutions, alcohol's, polyethylene glycol's, polyhydroxyethoxylated castor oil, peanut oil, olive oil, lactose, terra alba, sucrose, cyclodextrin, amylose, magnesium stearate, talc, gelatine, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and olyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed. release of the active ingredient after administration to the patient by employing procedures well known in the art.

The pharmaceutical compositions can be sterilized and mixed, if desired, with auxiliary agents, emulsifiers, salt for influencing osmotic pressure, buffers and/or colouring substances and the like, which do not deleteriously react with the active ingredient.

The route of administration may be any route, which effectively transports the active ingredient to the appropriate or desired site of action, such as oral, nasal, pulmonary, transdermal or parenteral e.g. rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatine capsule in powder or pellet form or it can be in the form of a troche or lozenge. If a liquid carrier is used, the preparation may be in the form of a syrup, emulsion, soft gelatine capsule or sterile injectable liquid such as an aqueous or non-aqueous liquid suspension or solution.

For nasal administration, the preparation may contain the active ingredient of the present invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

For parenteral application, particularly suitable are injectable solutions or suspensions, preferably aqueous solutions with the active ingredient dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet, which may be prepared by conventional tableffing techniques may contain:

| | |
|---|---|
| Active ingredient | 5.0 mg |
| Lactosum | 67.8 mg Ph.Eur. |
| Avicel ® | 31.4 mg |
| Amberlite ® | 1.0 mg |
| Magnesii stearas | 0.25 mg Ph.Eur. |

The present polymorphic/pseudopolymorphic forms of 6-chloro-3-(1-methylcyclopropyl)amino-4H-thieno[3,2-e]-1,2,4-thiadiazine 1,1-dioxide or mixtures thereof may be administered to a mammal, especially a human in need of such treatment, prevention, elimination, alleviation or amelioration of diseases related to the regulation of blood sugar. Such mammals include also animals, both domestic animals, e.g. household pets, and non-domestic animals such as wildlife.

The polymorphic/pseudopolymorphic forms of 6-chloro-3-(1-methylcyclopropyl)-amino-4H-thieno[3,2-e]-1,2,4-thiadiazine 1,1-dioxide or mixtures thereof are effective over a wide dosage range. A typical oral dosage is in the range of from about 0.001 to about 100 mg/kg body weight per day, e.g. from about 0.01 to about 50 mg/kg body weight per day, or e.g. from about 0.05 to about 20 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain of from 0.05 to about 1000 mg, e.g. from about 0.1 to about 500 mg, or e.g. from about 0.5 mg to about 200 mg.

The polymorphic/pseudopolymorphic forms of 6-chloro-3-(1-methylcyclopropyl)-amino-4H-thieno[3,2-e]-1,2,4-thiadiazine 1,1-dioxide or mixtures thereof were prepared synthesized as described in the following examples, which however, are not to be construed as limiting the scope of protection. The features disclosed in the foregoing description and in the following examples may, both separately and in any combination thereof, be material for realising the invention in diverse forms thereof.

Any novel feature or combination of features described herein is considered essential to this invention.

### EXAMPLES

The structures of the compounds are confirmed by either elemental analysis and/or proton Nuclear Magnetic Resonance (¹H-NMR) Spectroscopy. The ¹H-NMR spectra have been recorded on a Bruker Avance 400 MHz magnetic field spectrometer. For the polymorphs positions of all peaks in the proton spectra relating to 6-chloro-3-(1-methylcyclopropyl)amino-4H-thieno[3,2-e]-1,2,4-thiadiazine 1,1-dioxide are given. For the pseudopolymorphs/solvates in addition peaks relating to the solvent are given. Chemical shifts (δ) are given in parts per million (ppm), while coupling constants (J) are given in Hz and rounded off to nearest integer or half-integer.

Mp is melting point as detected by Differential Scanning Calorimetry (DSC), and is given in ° C. The DSC analysis is an enthalpy-change method in which the difference in energy inputs into a substance and a reference material is measured as a function of temperature by which a thermogram is recorded. The thermic events in the sample are reflected by the thermogram where an endothermic peak (the sample is absorbing heat) can be a solid solid state transition or a melting of the substance. An exotherm peak means that the sample is giving off heat and the underlying process can be a recrystallization or a simple degradation/combustion.

Weight loss as a function of the applied temperature is detected by the thermogravimetric analysis (TGA). In this technique the weight of a sample is monitored by an instrument that is equipped with a programmed temperature increase. The TGA analysis is particularly suited to the study of desolvation of a pseudopolymorph, as the release of the solvate will lead to a characteristic shift of the thermogravimetric weight loss temperature profile. The combination of TGA studies with DSC work can lead to an unambiguous assignment of the cause of observed thermal events.

A crystal is a solid state material whose atomic structure is periodic in three dimensions. The crystallinity is measured by powder X-ray diffraction (PXRD). The technique is based upon the interaction between electrons and X-ray radiation. In a crystal there are an infinite number of sets of lattice planes covering different directions in physical space. The X-ray radiation is diffracted by the electrons of the atoms at the lattice planes and each set of parallel planes gives rise to a peak in the diffraction pattern. The intensity of the diffracted radiation is measured as a function of scattering angle 2θ by which a diffractogram is recorded. A higher scattering angle corresponds to a smaller spacing between the lattice planes in the set. The position of the peaks is related to the physical dimensions of the crystal building stone, the unit cell, whereas the peak heights, i.e. the intensities are related to the arrangement and type of atoms residing on the set of planes in question. A chemical compound may exist in different crystalline forms each with a characteristic three-dimensional atomic arrangement. These are called polymorphs. Each polymorph will have a unique X-ray diffraction pattern (diffractogram) and the technique is therefore widely used in the analysis of polymorphism. The reported powder diffractograms have been recorded using CuK_{α} radiation, wavelength λ=1.5418 Å in reflection geometry using a flat plate sample holder.

Column chromatography was carried out using the technique described by W. C. Still et al, J. Org. Chem. 1978, 43, 2923-2925 on Merck silica gel 60 (Art 9385).

Compounds used as starting materials are either known compounds or compounds, which can readily be prepared by methods known per se.

Abbreviations:

| | |
|---|---|
| THF: | tetrahydrofurane |
| DMSO: | dimethylsulfoxide |
| CDCl₃: | deuterated chloroform |
| MgSO₄: | magnesium sulphate |
| sec: | seconds |
| min: | minutes |
| h: | hours |
| NMR: | Nuclear Magnetic Resonance |
| s: | singlet peak (in ¹H-NMR spectra) |
| d: | doublet of peaks (in ¹H-NMR spectra) |
| t: | triplet of peaks (in ¹H-NMR spectra) |
| q: | quartet of peaks (in ¹H-NMR spectra) |
| p: | penta (5) quintet of peaks (in ¹H-NMR spectra) |
| br. s: | broad singlet peak (in ¹H-NMR spectra) |
| DSC: | Differential Scanning Calorimetry |
| PXRD: | Powder X-ray Diffraction |
| Anal: | Elemental analysis |

### Example 1

Process for the Preparation of 6-chloro-3-(1-methylcyclopropyl)amino-4H-thieno[3,2-e]-1,2,4-thiadiazine 1,1-dioxide of Formula (I)

The compound is prepared as described in Example 3 of PCT application WO 00/37474, the contents of which are hereby incorporated by reference:

Method 1

A solution of 3,6-dichloro-4H-thieno[3,2-e]-1,2,4-thiadiazine 1,1-dioxide (386 mg, 1.5 mmol) in 1-methylcyclopropylamine (1.0 g, 14 mmol) was stirred for 24 h at 85° C. in a sealed flask. The cooled solution was concentrated in vacuo and the residue was stirred with ethyl acetate (1-2 mL) and filtered. The white precipitate was stirred in 4M hydrochloric acid (5 mL) for 2 h and then filtered off and chromatographed on silica gel with ethyl acetate to give 112 mg (26%) of the pure title compound; mp 251-252° C. dec; ¹H-NMR (DMSO-d₆): δ 0.65-0.79 (m, 4H), 1.36 (s, 3H), 7.11 (s, 1H), 7.82 (br s,1H), 10.78 (br s,1H); MS: m/e 291/293 (M+). An ethyl acetate solvate designated form C with an ethyl acetate content of 6.9% w/w was formed. The molar ratio of ethyl acetate to 6-chloro-3-(1-methylcyclopropyl)amino-4H-thieno[3,2-e]-1,2,4-thiadiazine 1,1-dioxide was established by ¹H-NMR spectroscopy to be 0.23 to 1.0.

### Example 2

Process for the Preparation of Polymorph A of 6-chloro-3-(1-methylcyclopropyl)-amino-4H- thieno[3,2-e]-1,2,4-thiadiazine 1,1-dioxide

Method 1

To a solution of 1 mL acetic acid is added 30 mg of 6-chloro-3-(1-methylcyclopropyl)amino-4H- thieno[3,2-e]-1,2,4-thiadiazine 1,1-dioxide. The mixture is heated to the boiling point, filtered and the filtrate allowed to stand for at least 24 h at 40° C. or room temperature. After a few days of evaporation, crystals designated form A are harvested. ¹H-NMR (DMSO-d₆): δ 0.69-0.75 (m, 4H), 1.36 (s, 3H), 7.11 (s, 1H), 7.80 (br.s,1H), 10.81 (brs, 1H).

Method 2

To a solution of 1 mL 2-propanol is added 30 mg of 6-chloro-3-(1-methylcyclopropyl)amino-4H- thieno[3,2-e]-1,2,4-thiadiazine 1,1-dioxide. The mixture is heated to the boiling point, filtered and the filtrate allowed to stand for at least 24 h at 40° C. or room temperature. After a few days of evaporation, crystals of form A are harvested.

Method 3

10 g of 6-chloro-3-(1-methylcyclopropyl)amino-4H- thieno[3,2-e]-1,2,4-thiadiazine 1,1-dioxide was suspended in 175 mL methanol and water. The mixture was heated to reflux while stirring. The mixture became clear and was subsequently treated with 1 g active carbon and filtered while keeping the temperature just below boiling point. The filter cake was washed with 10 mL of a mixture of 90% methanol and 10% water. To the filtrate was added 100 mL water at reflux while stirring. The heating bath was removed and the mixture was allowed to cool to room temperature. The mixture was then put on an ice bath and cooled to 5° C. while stirring. After 2 h at 5° C. the suspension was filtered and the filter cake washed with 10 mL methanol. After drying, 9.0 g of 6-chloro-3-(1-methylcyclopropyl)amino-4H-thieno[3,2-e]-1,2,4-thiadiazine 1,1-dioxide crystal form A was obtained.

Method 4

10 g of 6-chloro-3-(1-methylcyclopropyl)amino-4H- thieho[3,2-e]-1,2,4-thiadiazine 1,1-dioxide was suspended in 100 mL 1-propanol and heated to reflux (95° C.) while stirring. At reflux, 15 mL water was added slowly. The solution became clear. The mixture was slightly cooled. At 80° C. the clear solution was treated with 1 g active carbon and heated again to reflux. After 1 h, the mixture is filtered. The heating bath was removed and the mixture was allowed to equilibrate to room temperature. The mixture was then put on a ice bath and cooled to 5° C. while stirring. After 2 h at 5° C. the suspension was filtered and the filter cake washed with 10 mL 1-propanol. After drying, 8.3 g of 6-chloro-3-(1-methylcyclopropyl)-amino-4H- thieno[3,2-e]-1,2,4-thiadiazine 1,1-dioxide crystal form A was obtained.

Method 5

15 g of 6-chloro-3-(1-methylcyclopropyl)amino-4H- thieno[3,2-e]-1,2,4-thiadiazine 1,1-dioxide was suspended in 150 mL 2-propanol and 23.5 mL water. While stirring, the mixture was heated to reflux and stirred until the mixture became clear. 1.5 g of active carbon was the added and reflux is maintained for another 1 h. The mixture was filtered on a pre-heated filter and the filter cake washed with a mixture of 9 mL 2-propanol and 1 mL water. 150 mL water was slowly added to the 75-80° C. hot filtrate while stirring. At 73° C. the crystallisation started. The mixture is slowly cooled to 5° C. and stirred for another 2 h at 5° C. The mixture was filtered and the filter cake washed with 2×10 mL 2-propanol. After drying, 12.2 g of 6-chloro-3-(1-methylcyclopropyl)amino-4H- thieno[3,2-e]-1,2,4-thiadiazine 1,1-dioxide crystal form A was obtained.

Method 6

In the first reactor 15 g of 6-chloro-3-(1-methylcyclopropyl)amino-4H- thieno[3,2-e]-1,2,4-thiadiazine 1,1-dioxide was suspended in 150 mL 2-propanol and 23.5 mL water while stirring. The suspension was heated to reflux. Heating was maintained until a clear solution was obtained. The solution was slightly cooled to below reflux temperature. 1.5 g activated carbon was added and the mixture refluxed for further 1 h. After 1 h, the solution was filtered on a preheated filter. The temperature of the filtrate was kept above 75° C. In a second reactor 50 mL water was preheated to 35° C. while stirring. 100 mL of water and the hot solution from reactor 1 was put into two different dropping funnels and added drop-wise to the second reactor containing the 50 mL water while stirring. The 100 mL water and the hot solution from reactor 1 were added in such a way that the addition was finished at the same time. The temperature in the second reactor was maintained at 35-45° C. After addition, the mixture was cooled to 20-25° C. and stirred further 2 h to complete the crystallisation. The suspension was filtered and the filter cake washed with 2×10 mL 2-propanol. After drying, 12,3 g of 6-chloro-3-(1-methylcyclopropyl)amino-4H- thieno[3,2-e]-1,2,4-thiadiazine 1,1-dioxide crystal form A was obtained.

Method 7

15 g of 6-chloro-3-(1-methylcyclopropyl)amino-4H- thieno[3,2-e]-1,2,4-thiadiazine 1,1-dioxide was suspended in 175 mL methanol and 100 mL n-butylacetate. The suspension was heated to reflux and methanol was added in portions until the solution became clear. In total 115 mL methanol was added while stirring. The mixture was slightly cooled and 2 g of active carbon was added. After 1 h at reflux, the solution was filtered. The solution was distilled thereby removing methanol. At 70° C. 100 mg seeding crystals of 6-chloro-3-(1-methylcyclopropyl)amino-4H- thieno[3,2-e]-1,2,4-thiadiazine 1,1-dioxide form A were added. The rest of the methanol was then removed by distillation over 45 min. During distillation, the reactor temperature was not allowed to exceed 92° C. The heating bath was removed and the mixture allowed cooling to room temperature while stirring. After 1 h at room temperature, the mixture was filtered and the filter cake washed with 30 mL n-butylacetate. After drying to constant weight, 14.5 g of 6-chloro-3-(1-methylcyclopropyl)amino-4H- thieno[3,2-e]-1,2,4-thiadiazine 1,1-dioxide crystal form A was obtained.

Method 8

149 g 6-chloro-3-(1-methylcyclopropyl)amino-4H- thieno[3,2-e]-1,2,4-thiadiazine 1,1-dioxide was dissolved in 745 mL 1-methyl-pyrrolidinone at room temperature, and the resulting solution was heated to 95° C. Then 1490 mL water was added drop wise, and the solution was cooled to room temperature over night followed by cooling on an ice bath for 1 h. A precipitate was observed. The precipitate was isolated by filtration, and the filter cake was washed with 3×300 mL water. After drying to constant weight, 140 g (94%) of 6-chloro-3-(1-methylcyclopropyl)amino-4H- thieno[3,2-e]-1,2,4-thiadiazine 1,1-dioxide crystal form A was obtained.

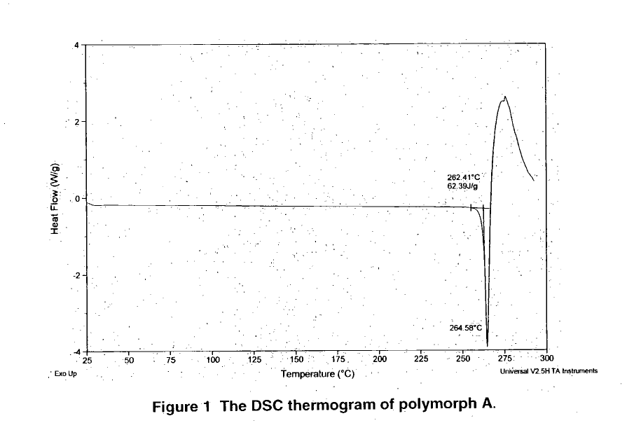

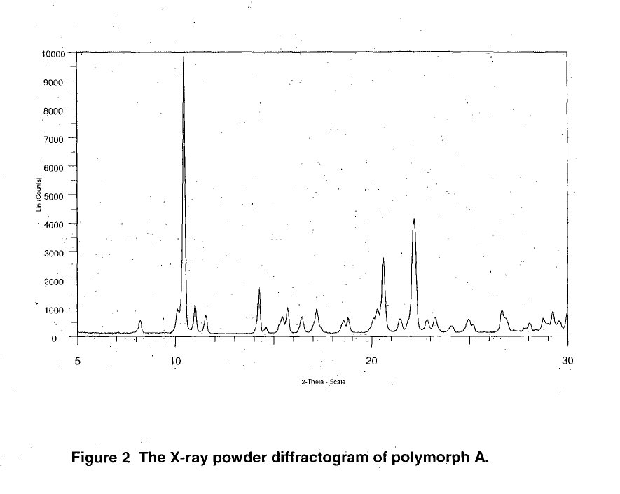

The polymorph A is characterised by having a DSC thermogram as shown in FIG. 1 and a powder X-ray diffractogram (PXRD) as shown in FIG. 2. The unit cell is triclinic, space group P-1 (no. 2), dimensions a=9.4443(6) Å,b=11.1933(8) Å, c=13.3000(11) Å, α=91.14(2)°, β=110.89(2)°, and γ=107.22(2)° (all at 120 K). The crystal density is by crystal structure determination calculated to 1.560 g/cm³ (at 120 K) while the experimental value determined by He-pychnometry is 1.546 g/cm³ (at 295 K). The melting point of polymorph A is 262.4° C. (onset).

### Example 3

Process for the Preparation of Polymorph B of 6-chloro-3-(1-methylcyclopropyl)-amino-4H- thieno[3,2-e]-1,2,4-thiadiazine 1,1-dioxide

Method 1

To a solution of 1 mL methanol is added 30 mg of 6-chloro-3-(1-methylcyclopropyl)-amino-4H- thieno[3,2-e]-1,2,4-thiadiazine 1,1-dioxide. The mixture is heated to the boiling point, filtered and the filtrate allowed to stand for at least 24 h at 40° C. After a few days of evaporation at 40° C. or higher temperature, crystals designated form B are harvested. ¹H-NMR (DMSO-d₆): δ 0.63-0.81 (m, 4H), 1.36 (s, 3H), 7.12 (s, 1H), 7.82 (br s, 1H), 10.74 (br s, 1H).

Method 2

To a solution of 1 mL ethanol is added 30 mg of 6-chloro-3-(1-methylcyclopropyl)-amino-4H- thieno[3,2-e]-1,2,4-thiadiazine 1,1-dioxide. The mixture is heated to the boiling point, filtered and the filtrate allowed to stand for at least 24.h at 40° C. After a few days of evaporation at 40° C. or higher temperature, crystals of form B are harvested.

Method 3

15 g of 6-chloro-3-(1-methylcyclopropyl)amino-4H- thieno[3,2-e]-1,2,4-thiadiazine 1,1-dioxide was suspended in 175 mL methanol and 100 mL n-butylacetate. The suspension was heated to reflux and methanol was added in portions until the solution became clear. In total 115 mL methanol was added while stirring. The mixture was slightly cooled and 2 g of active carbon was added. After 1 h at reflux, the solution is filtered. The solution is distilled over 3 h thereby removing methanol. During distillation, the reactor temperature did not exceed 92° C. The heating bath is removed and the mixture is allowed to cool to room temperature while stirring. After 1 h at room temperature, the mixture was filtered and the filter cake washed with n-butylacetate. After drying to constant weight, 14 g of 6-chloro-3-(1-methylcyclopropyl)amino-4H- thieno[3,2-e]-1,2,4-thiadiazine 1,1-dioxide crystal modification B was obtained.

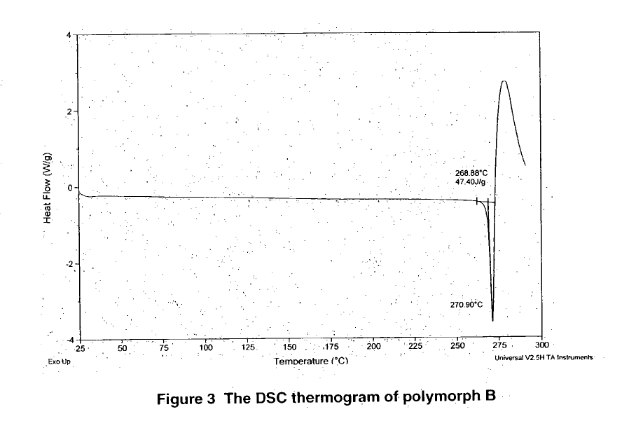

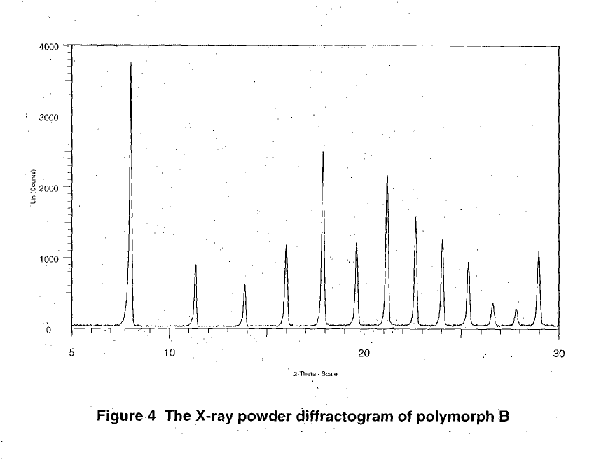

The polymorph B is characterised by having a DSC thermogram as shown in FIG. 3 and a powder X-ray diffractogram (PXRD) as shown in FIG. 4. The unit cell is rhombohedral, space group R-3 (no.148), dimensions a=b=22.24 Å, c=13.62 Å, α=β=90°, and γ=120° (at 295 K). The crystal density of this polymorph is by crystal structure determination calculated to 1.494 g/cm³, while the experimental value determined by He-pychnometry is 1.499 g/cm³ (both at 295 K). The melting point of polymorph B is 268.9° C. (onset).

### Example 4

Process for the Preparation of Pseudopolymorph C1 of 6-chloro-3-(1-methylcyclopropyl)amino-4H- thieno[3,2-e]-1,2,4-thiadiazine 1,1-dioxide, a Solvate of Acetone

Method 1

To a solution of 1 mL acetone is added 15 mg of 6-chloro-3-(1-methylcyclopropyl)-amino-4H- thieno[3,2-e]-1,2,4-thiadiazine 1,1-dioxide. The mixture is heated to the boiling point, filtered and the filtrate allowed to stand for at least 24 h at 40° C. After a few days of evaporation at room temperature or higher temperature, crystals designated form C1, a solvate with 4.6% w/w acetone, are harvested. ¹H-NMR (DMSO-d₆): δ 0.65-0.85 (m, 4H), 1.36 (s, 3H), 7.12 (s,1H), 7.81 (br s, 1H), 10.75 (br s,1H), solvent: δ 2.09 (s). The molar ratio of acetone to 6-chloro-3-(1-methylcyclopropyl)amino-4H- thieno[3,2-e]-1,2,4-thiadiazine 1,1-dioxide was established by ¹H-NMR spectroscopyy to be 0.24 to 1.0.

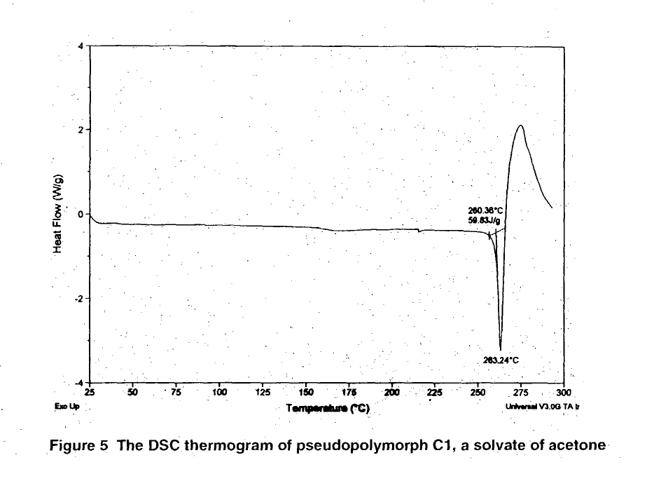

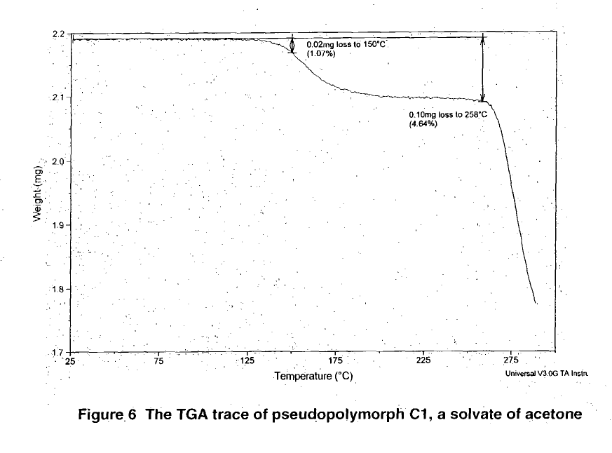

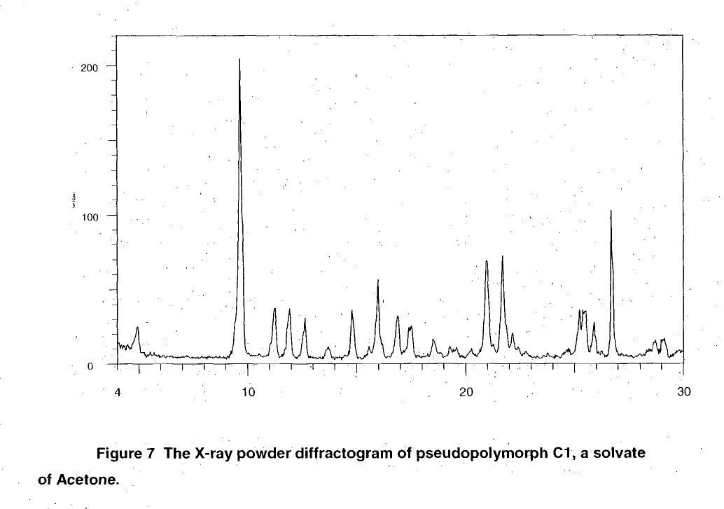

The pseudopolymorph C1 is characterised by having a DSC thermogram as shown in FIG. 5; a TGA trace as shown in FIG. 6 and a powder X-ray diffractogram (PXRD) as shown in FIG. 7. The melting point of polymorph C1 from acetone is 260.4° C. (onset).

### Example 5

Process for the Preparation of Pseudopolymorph C2 of 6-chloro-3-(1-methylcyclopropyl)amino-4H- thieno[3,2-e]-1,2,4-thiadiazine 1,1-dioxide, a solvate of 1-butanol

Method 1

10 g of 6-chloro-3-(1-methylcyclopropyl)amino-4H- thieno[3,2-e]-1,2,4-thiadiazine 1,1-dioxide was suspended in 100 mL 1-butanol and heated to reflux while stirring. During reflux 9 mL water is added and the mixture becomes clear. The mixture is slightly cooled to 85° C. and 1 g of active carbon is added. The mixture is heated to ref lux again. After 1 h the mixture was filtrated. To the hot filtrate (95-100° C.) was added 50 mL water while stirring. At 91° C. and 35 mL water added the solution becomes cloudy. To liquid phases are formed as expected. The remaining 15 mL of water is added slowly and the mixture is allowed to cool to room temperature. At 40° C., the crystallisation process starts. The mixture is further cooled to 5° C. and after 2 h filtrated. The filter cake is washed and 6.35 g of vacuum dried (at 100° C.) 6-chloro-3-(1-methylcyclopropyl)amino-4H- thieno[3,2-e]-1,2 4-thiadiazine 1,1-dioxide designated crystal form C2, a solvate with 6.3% w/w 1-butanol, are harvested. ¹H-NMR (DMSO-d6): δ 0.65-0.80 (m, 4H), 1.36 (s, 3H), 7.12 (s, 1H), 7.80 (br s, 1H), 10.74 (br s, 1H), solvent: δ 0.86 (t, 3H, J=7 Hz),1.30 (m, 2H), 1.39 (m, 2H,) 3.38 (m, 2H), 4.33 (t, 1H, J=5 Hz). The molar ratio of 1-butanol to 6-chloro-3-(1-methylcyclopropyl)amino-4H- thieno[3,2-e]-1,2,4-thiadiazine 1,1-dioxide was established by ¹H-NMR spectroscopy to be 0.25 to 1.0.

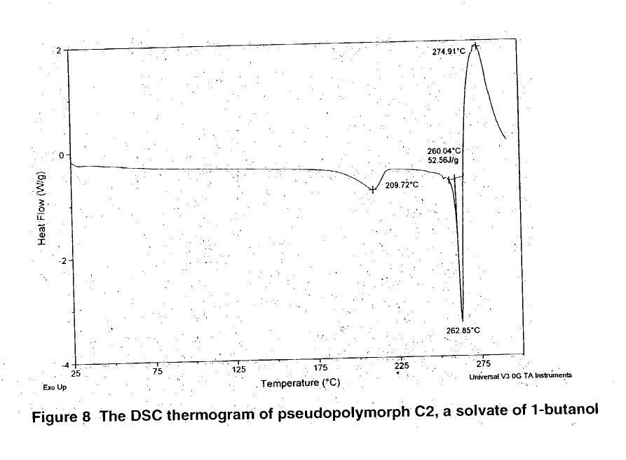

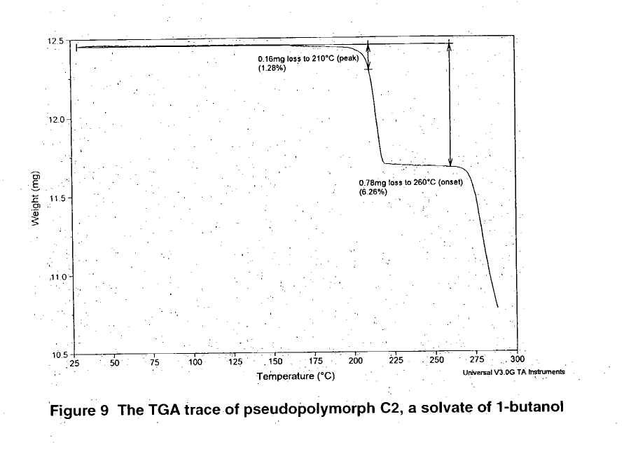

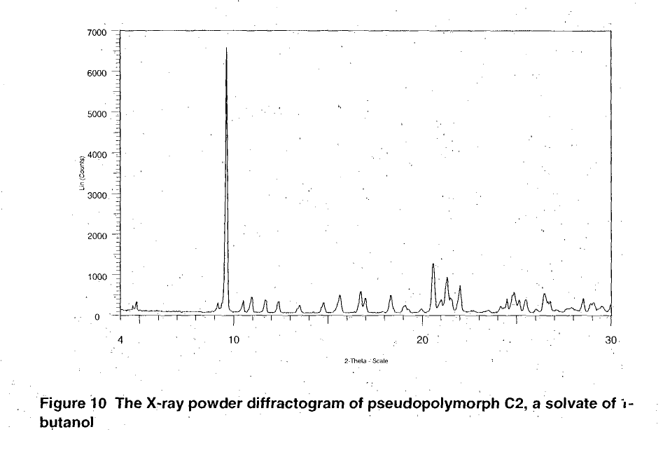

The pseudopolymorph C2 is characterised by having a DSC thermogram as shown in FIG. 8; a TGA trace as shown in FIG. 9 and a powder X-ray diffractogram (PXRD) as shown in FIG. 10. The melting point of polymorph C2 from 1-butanol is 260.0° C. (onset).

### Example 6

Process for the Preparation of Pseudopolymorph C3 of 6-chloro-3-(1-methylcyclopropyl)amino-4H- thieno[3,2-e]-1,2,4-thiadiazine 1,1-dioxide, a Solvate of 2-butanol

Method 1

To a solution of 2 mL 2-butanol is added 30 mg of 6-chloro-3-(1-methylcyclopropyl)-amino-4H- thieno[3,2-e]-1,2,4-thiadiazine 1,1-dioxide. The mixture is heated to the boiling point, the suspension is filtered and the filtrate allowed to stand for at least 24 h at 40° C. After a few days of evaporation at room temperature or higher temperature, crystals designated form C3, a solvate with 6.4% w/w 2-butanol, are harvested. ¹H-NMR (DMSO-d₆): δ 0.65-0.80 (m, 4H), 1.36 (s, 3H), 7.12 (s, 1H), 7.82 (br s, 1H), 10.74 (br s, 1H), solvent: δ 0.83 (t, 3H, J=7.5 Hz), 1.02 (d, 3H, J=6.0 Hz, 1.33 (m, 2H), 3.49 (septet, 1H, J=6.0 Hz), 4.32 (d, 1H, J=4.5 Hz). The molar ratio of 2-butanol to 6-chloro-3-(1-methylcyclopropyl)amino-4H-thieno[3,2-e]-1,2,4-thiadiazine 1,1-dioxide was established by ¹H-NMR spectroscopy to be 0.25 to 1.0.

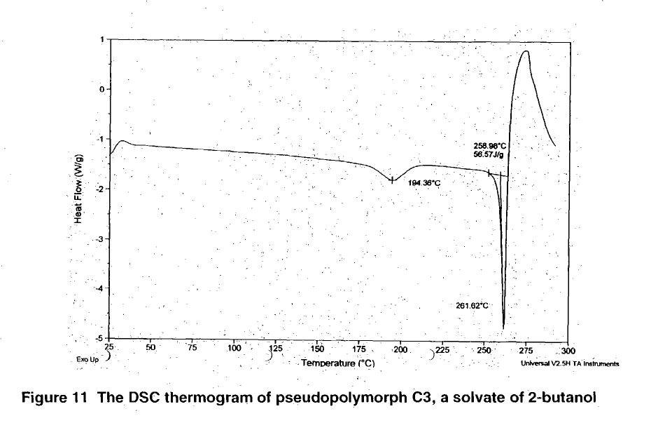

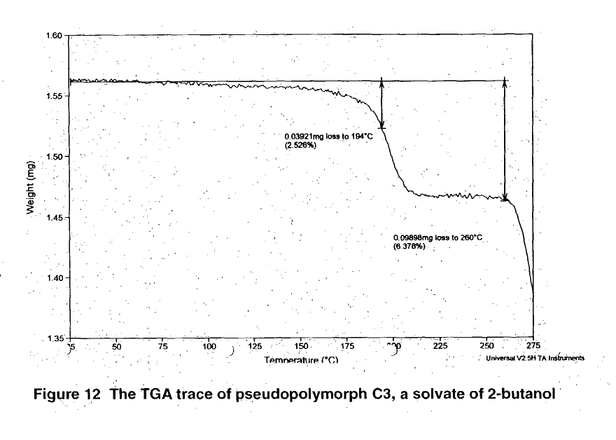

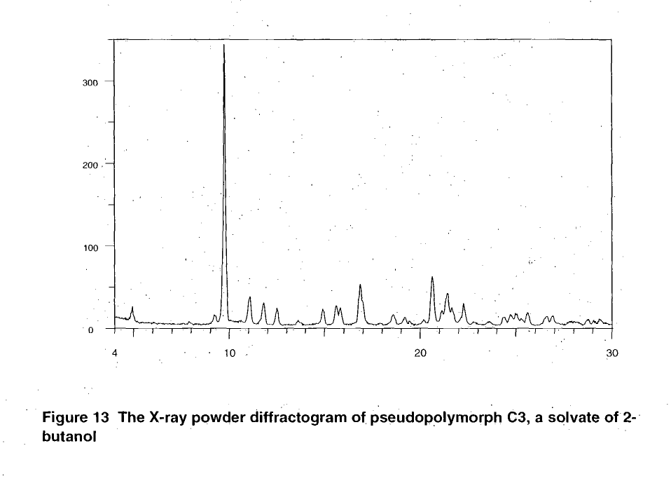

The pseudopolymorph C3 is characterised by having a DSC thermogram as shown in FIG. 11; a TGA trace as shown in FIG. 12 and a powder X-ray diffractogram (PXRD) as shown in FIG. 13. The melting point of polymorph C3 from 2-butanol is 259.0° C. (onset).

### Example 7

Process for the Preparation of Pseudopolyrnorph C4 of 6-chloro-3-(1-methylcyclopropyl) amino-4H- thieno[3,2-e]-1,2,4-thiadiazine 1,1-dioxide, a Solvate of 1,4-dioxane

Method 1

To a solution of 1,8 mL 1,4-dioxane was added 33 mg of 6-chloro-3-(1-methylcyclopropyl)amino-4H- thieno[3,2-e]-1,2,4-thiadiazine 1,1-dioxide. The mixture was heated to the boiling point, and the suspension was allowed to stand for at least 24 h at 40° C. After a few days of evaporation at room temperature or higher temperature, the powder crystalline sample of polymorph form designated C4, a solvate with 7.4% w/w of 1,4-dioxane was harvested. ¹H-NMR (DMSO-d₆): δ 0.65-0.80 (m, 4H), 1.36 (s, 3H), 7.12 (s, 1H), 7.83 (br s, 1H), 10.74 (br s,1H), solvent: δ 3.57 (s). The molar ratio of 1,4-dioxane to 6-chloro-3-(1-methylcyclopropyl)amino-4H-thieno[3,2-e]-1,2,4-thiadiazine 1,1-dioxide was established by ¹H-NMR spectroscopy to be 0.25 to 1.0.

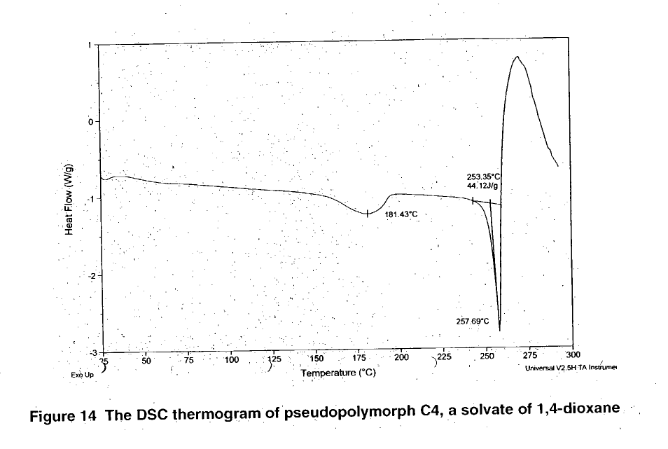

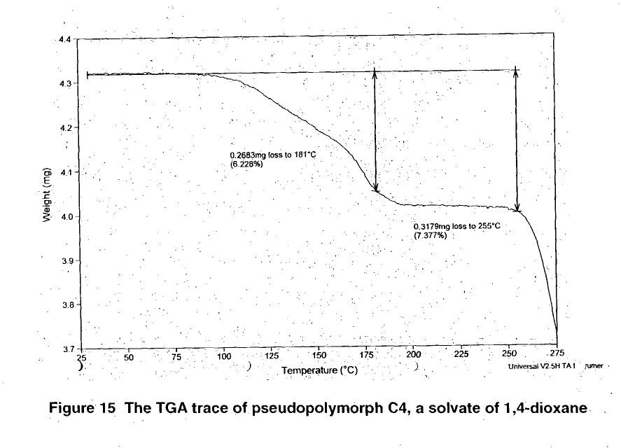

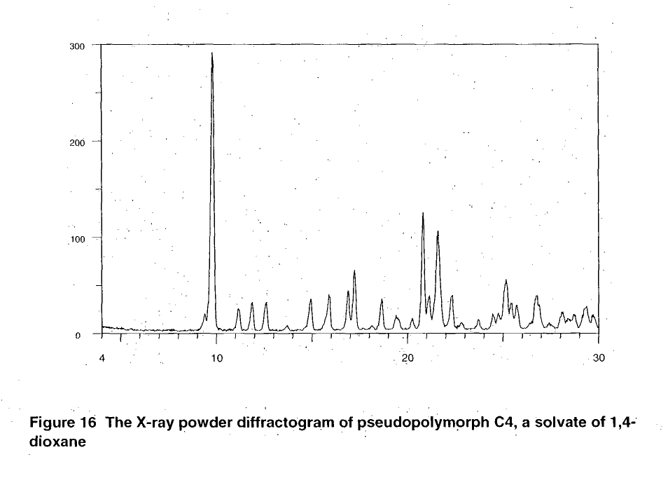

The pseudopolymorph C4 is characterised by having a DSC thermogram as shown in FIG. 14; a TGA trace as shown in FIG. 15 and a powder X-ray diffractogram (PXRD) as shown FIG. 16. The melting point of polymorph C4 from 1,4-dioxane is 253.4° C. (onset).

### Example 8

Process for the Preparation of Pseudopolymorph C5 of 6-chloro-3-(1-methylcyclopropyl)amino-4H- thieno[3,2-e]-1,2,4-thiadiazine 1,1-dioxide, a Solvate of Methylacetate

Method 1

To a solution of 1.0 mL methylacetate is added 35 mg of 6-chloro-3-(1-methylcyclopropyl)amino-4H- thieno[3,2-e]-1,2,4-thiadiazine 1,1-dioxide. The mixture is heated to the boiling point, and the suspension allowed standing for at least 24 h at 40° C. After a few days the solvent is evaporated and the powder crystalline sample is harvested and left to dry. The crystals are designated form C5, a solvate with 5.2% w/w methylacetate. ¹H-NMR (DMSO-d₆): δ 0.65-0.80 (m, 4H), 1.36 (s, 3H), 7.12 (s, 1H), 7.82 (br s, 1H), 10.75 (br s, 1H), solvent: δ 2.01 (s, 3H), 3.57 (s, 3H). The molar ratio of methylacetate to 6-chloro-3-(1-methylcyclopropyl)amino-4H- thieno[3,2-e]-1,2,4-thiadiazine, 1,1-dioxide was established by NMR spectroscopy to be 0.19 to 1.0.

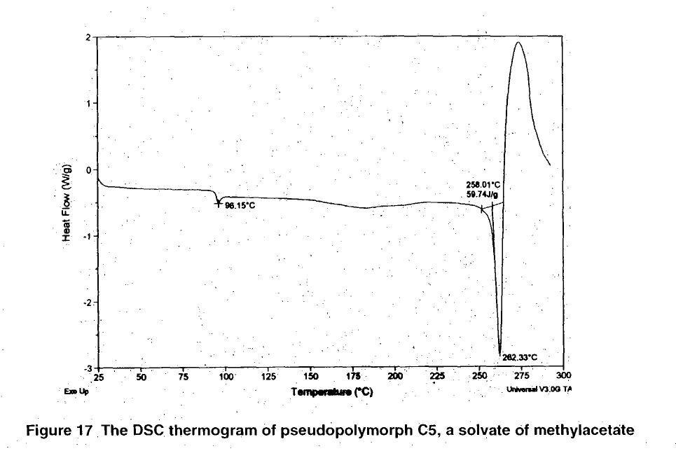

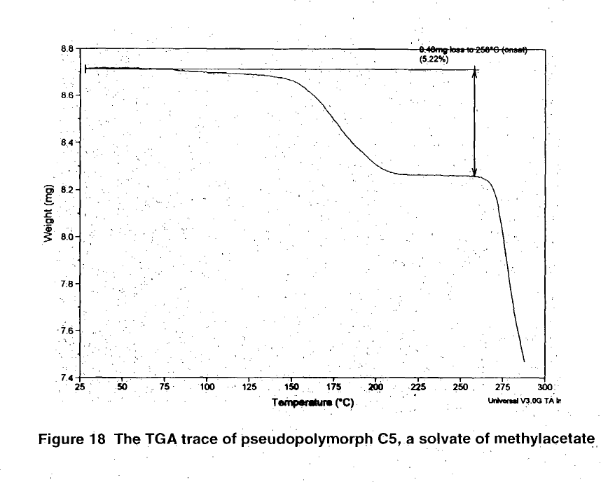

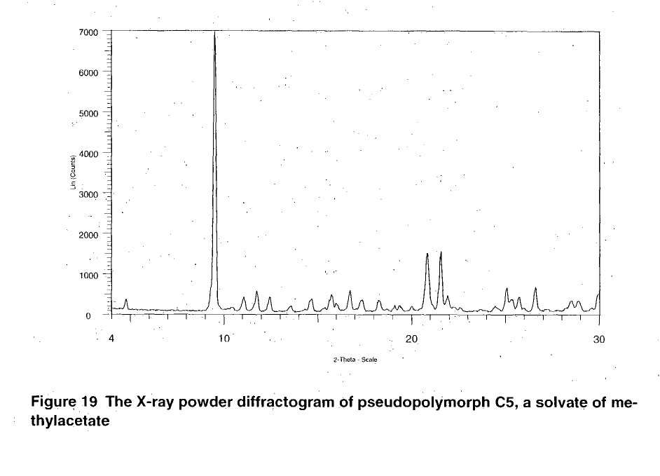

The pseudopolymorph C5 is characterised by having a DSC thermogram as shown in FIG. 17; a TGA trace as shown in FIG. 18 and a powder X-ray, diffractogram (PXRD) as shown in FIG. 19. The melting point of polymorph C5 from met hylacetate is 258.0° C. (onset).

### Example 9

Process for the Preparation of Pseudopolymorph C6 of 6-chloro-3-(1-methylcyclopropyl)amino-4H- thieno[3,2-e]-1,2,4-thiadiazine 1,1-dioxide, a Solvate of Methylethylketone (or MEK or 2-butanone)

Method 1

To a solution of 1.0 mL methylethylketone is added 35 mg of 6-chloro-3-(1-methylcyclopropyl)amino-4H- thieno[3,2-e]-1,2,4-thiadiazine 1,1-dioxide. The mixture is heated to the boiling point, and the suspension allowed standing for at least 24 h at 40° C. After a few days the mother liquor is removed and the powder crystalline sample is harvested and left to dry. The crystals are designated form C6, a solvate with 6.1% w/w methylethylketone. ¹H-NMR (DMSO-d₆): δ 0.65-0.80 (m, 4H), 1.36 (s, 3H), 7.12 (s,1H), 7.83 (br s, 1H), 10.75 (br s, 1H), solvent: δ 0.91 (t, 3H, J=7.0 Hz), 2.07 (s, 3H), 2.43 (q, 2H, J=7.0 Hz)). The molar ratio of methylethylketone to 6-chloro-3-(1-methylcyclopropyl)amino-4H-thieno[3,2-e]-1,2,4-thiadiazine 1,1-dioxide was established by NMR spectroscopy to be 0.23 to 1.0.

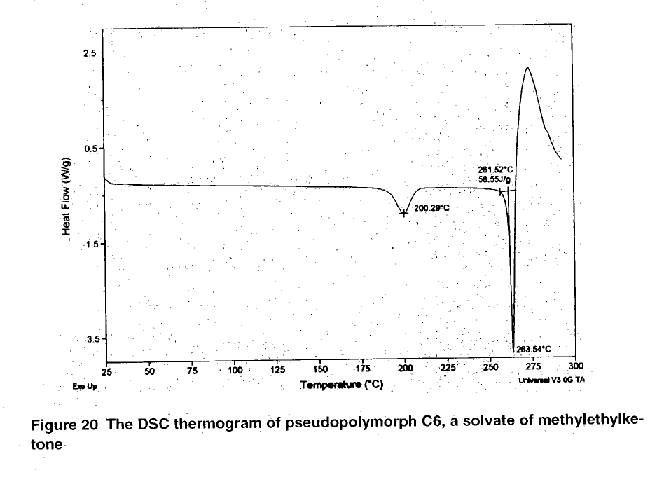

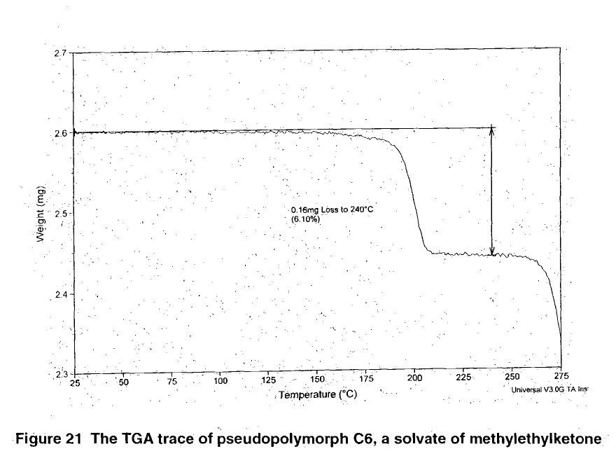

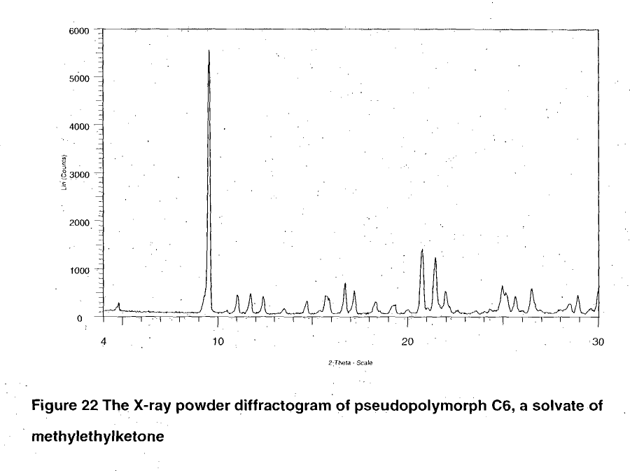

The pseudopolymorph C6 is characterised by having a DSC thermogram as shown in FIG. 20; a TGA trace as shown in FIG. 21 and a powder X-ray diffractogram (PXRD) as shown in FIG. 22: The melting point of polymorph C6 from methylethylketone is 261.5° C. (onset).

### Example 10

Process for the Preparation of Pseudopolymorph C7 of 6-chloro-3-(1-methylcyclopropyl)amino-4H- thieno[3,2-e]-1,2,4-thiadiazine 1,1-dioxide, a solvate of tetrahydrofurane (THF)

Method 1

To a solution of 1.6 mL THF is added 32mg of 6-chloro-3-(1-methylcyclopropyl)-amino-4H- thieno[3,2-e]-1,2,4-thiadiazine 1,1-dioxide. The mixture is heated to the boiling point, filtered and the filtrate allowed to stand for at least 24 h at 40° C. After a few days of evaporation at room temperature or higher temperature, crystals designated form C7, a solvate with 5.7% w/w THF, are harvested. ¹H-NMR (DMSO-d₆): δ 0.65-0.80 (m, 4H), 1.36 (s, 3H), 7.12 (s, 1H), 7.81 (br s, 1H), 10.74 (br s, 1H), solvent: 6 1.76 (m, 4H), 3.60 (m, 4H). The molar ratio of THF to 6-chloro-3-(1-methylcyclopropyl)amino-4H- thieno[3,2-e]-1,2,4-thiadiazine 1,1-dioxide was established by NMR spectroscopy to be 0.23 to 1.0.

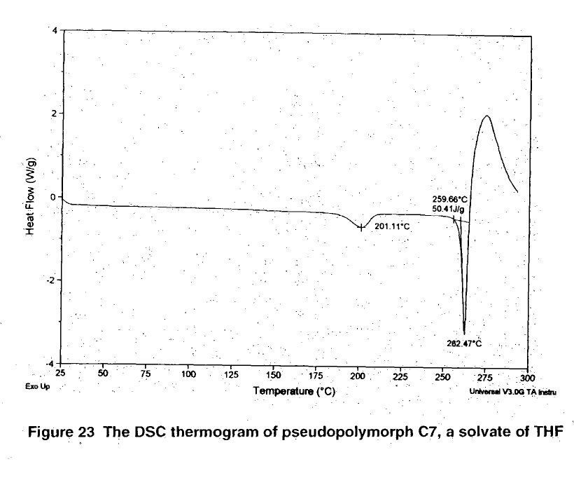

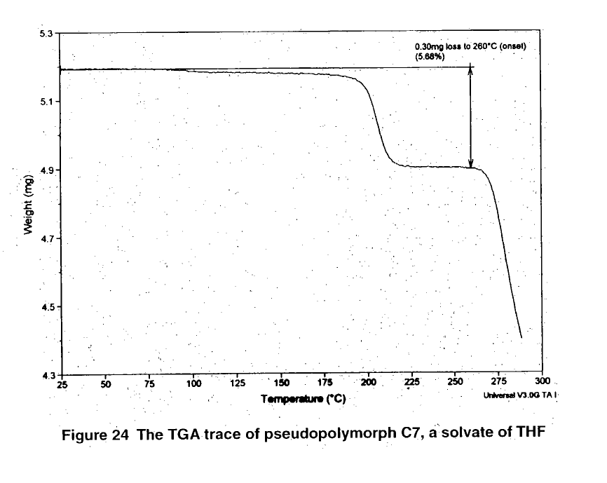

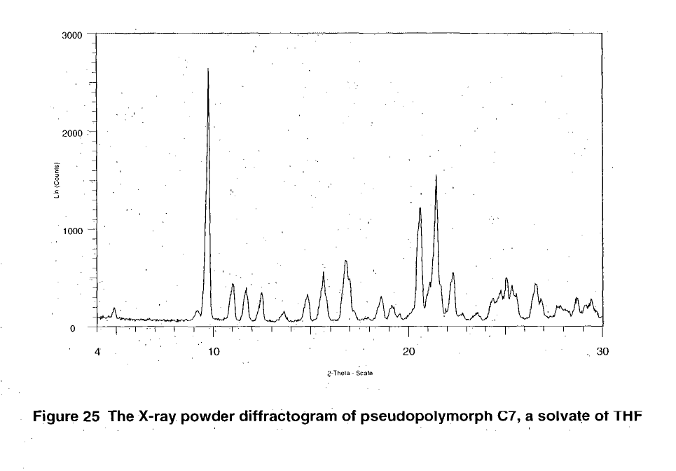

The pseudopolymorph C7 is characterised by having a DSC thermogram as shown in FIG. 23; a TGA trace as shown in FIG. 24 and a powder X-ray diffractogram (PXRD) as shown in FIG. 25. The melting point of polymorph C7 from THF is 259.7° C. (onset).

### Example 11

Process for the Preparation of Pseudopolymorph C8 of 6-chloro-3-(1-methylcyclopropyl)amino-4H- thieno[3,2-e]-1,2,4-thiadiazine 1,1-dioxide, a Solvate of Toluene

Method 1

To a solution of 2.0 mL toluene was added 32 mg of 6-chloro-3-(1-methylcyclopropyl)amino-4H-thieno[3,2-e]-1,2,4-thiadiazine 1,1-dioxide. The mixture was heated to the boiling point, and the suspension was allowed to stand for at least 24 h at 40° C. After a few days of evaporation at room temperature or higher temperature, the mother liquor was removed and powder crystals designated polymorph form C8, a solvate with 7.2% w/w of toluene was harvested. ¹H-NMR (DMSO-d₆): δ 0.63-0.79 (m, 4H), 1.36 (s, 3H), 7.11 (s,1H), 7.83 (br s,1H), 10.78 (br s, 1H), solvent: 6 2.30 (s, 3H), 7.15-7.29 (m, 5H) . The molar ratio of toluene to 6-chloro-3-(1-methylcyclopropyl)amino-4H- thieno[3,2-e]-1,2,4-thiadiazine 1,1-dioxide was established by NMR spectroscopy to be 0.26 to 1.0.

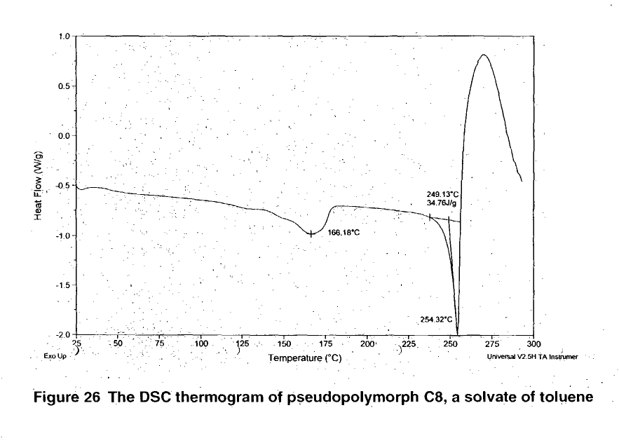

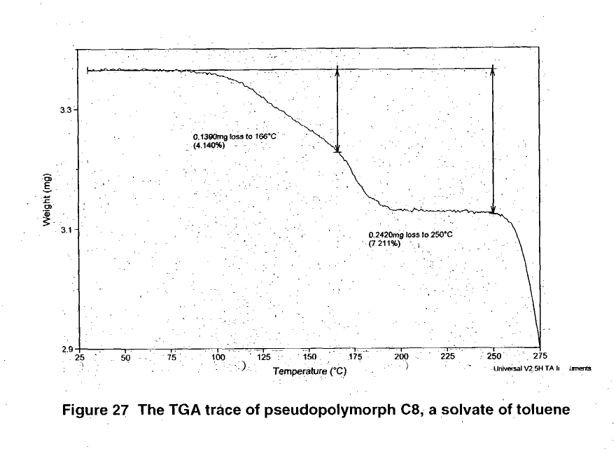

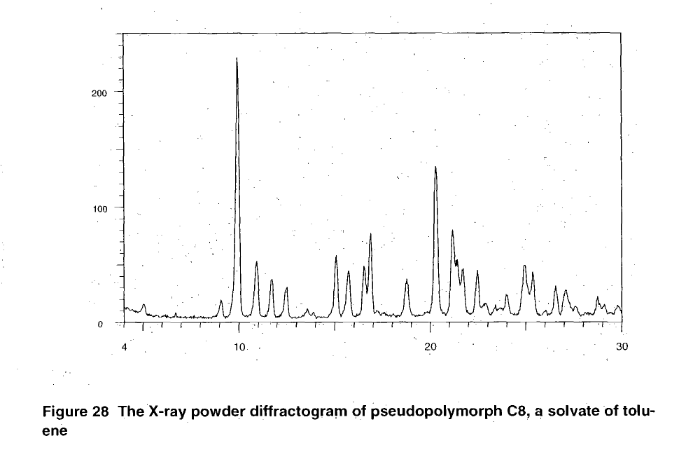

The pseudopolymorph C8 is characterised by having a DSC thermogram as shown in FIG. 26; a TGA trace as shown in FIG. 27 and a powder X-ray diffractogram (PXRD) as shown in FIG. 28. The melting point of polymorph C8 from toluene is 249.1° C. (onset).

### Example 12

Process for the Preparation of Pseudopolymorph C9 of 6-chloro-3-(1-methylcyclopropyl)amino-4H- thieno[3,2-e]-1,2,4-thiadiazine 1,1-dioxide, a Solvate of 1-pentanol

Method 1

To a solution of 1.0 mL 1-pentanol is added 36 mg of 6-chloro-3-(1-methylcyclopropyl)amino-4H- thieno[3,2-e]-1,2,4-thiadiazine 1,1-dioxide. The mixture is heated to the boiling point, and the suspension allowed to stand for at least 24 h at 40° C. After a few days of evaporation at room temperature or higher temperature, powder crystals designated polymorph form C9, a solvate with 7.1% w/w 1-pentanol, are harvested. ¹H-NMR (DMSO-d₆): δ 0.65-0.80 (m, 4H), 1.36 (s, 3H), 7.12 (s, 1H), 7.81 (br s, 1H), 10.74 (br s,1H), solvent: δ 0.86 (t, 3, J=7.0 Hz), 1.27 (m, 4H), 1.41 (p, 2H, J 7.0 Hz), 3.39 (m, 2H), 4.34 (t,1H, J=5.0 Hz). The molar ratio of 1-pentanol to 6-chloro-3-(1-methylcyclopropyl)amino-4H- thieno[3,2-e]-1,2,4-thiadiazine 1,1-dioxide was established by NMR spectroscopy to be 0.24 to 1.0.

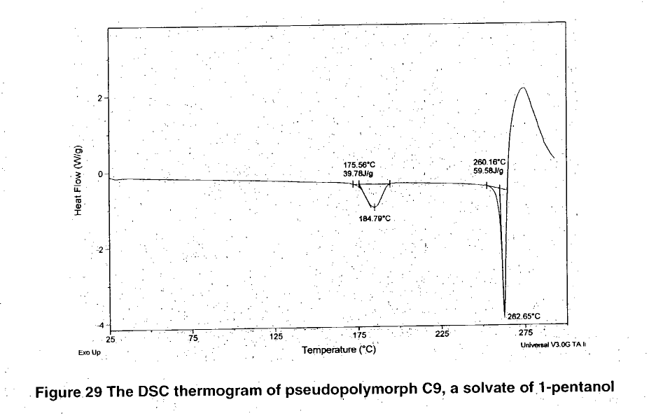

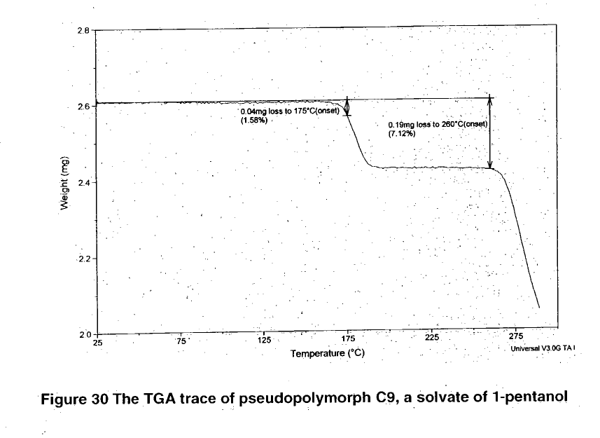

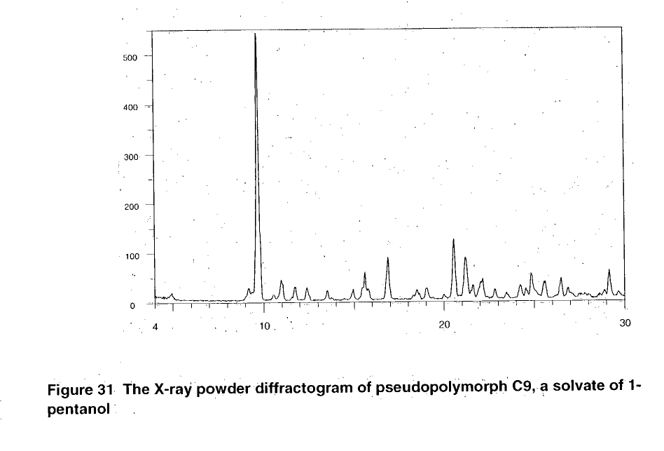

The pseudopolymorph C9 is characterised by having a DSC thermogram as shown in FIG. 29; a TGA trace as shown in FIG. 30 and a powder X-ray diffractogram (PXRD) as shown in FIG. 31. The melting point of polymorph C9 from 1-pentanol is 260.2° C. (onset).

### Example 13

Process for the Preparation of Polymorph D of 6-chloro-3-(1-methylcyclopropyl)amino-4H- thieno[3,2-e]-1,2,4-thiadiazine 1,1-dioxide

Method 1

In a nitrogen atmosphere 1.74 g KF is dissolved in 25 mL methanol at 35° C. while stirring. The resulting clear solution is evaporated to dryness and 16 mL NMP is added. Half of the added NMP is again distilled off under vacuum at 70° C. 8 mL NMP is added while flushing with nitrogen. To the white suspension is added 0.11 g cetyltrimethylammoniumbromide and 2.57 g 3,6-dichloro-4H-thieno[3,2-e]-1,2,4-thiadiazine 1,1-dioxide while flushing with nitrogen. The reactor is equipped with a condenser, N₂-addition, and a heating facility. The temperature is raised to 140° C. while stirring. 24 h later additional 0.11 g cetyltrimethylammoniumbromide is added. After 30 h additional pretreated (as mentioned above) 0.58 g KF is added. After 48 h the mixture is cooled to room temperature and 2.8 mL triethylamine and 1.61 g methylcyclopropylamine hydrochloride is added. The temperature is raised to 70° C. and the mixture is stirred for another 24 h at 70° C. After cooling a white precipitate is filtered off. The filter cake is washed with 2×2.5 mL NMP. The filtrate is added drop wise to a solution of 3.7 g sodium acetate, 1.15 mL acetic acid, and 50 mL water. The pH of the resulting mixture was measured to 12.2 after addition of the NMP solution. Additional 0.6 mL acetic acid is added. A white, greasy precipitate is formed and is filtered off. To the filtrate is added 0.9 mL acetic acid. The pH is now 4.5. Additional 25 mL water is added to the solution. A white precipitate is formed. The solution is cooled on an ice bath and filtered. After drying of the filter cake to constant weight, 1.41 g of 6-chloro-3-(1-methylcyclopropyl)amino-4H-thieno[3,2-e]-1,2,4-thiadiazine 1,1-dioxide crystals designated form D was obtained. ¹H-NMR (DMSO-d₆): δ 0.65-0.80 (m, 4H), 1.36 (s, 3H), 7.11 (s, 1H), 7.78 (br s,1H), 10.71 (br s,1H).

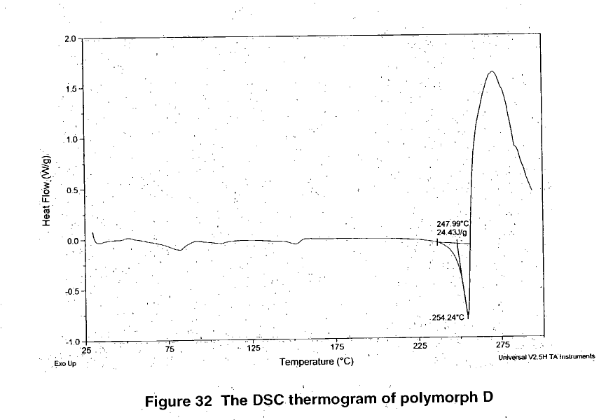

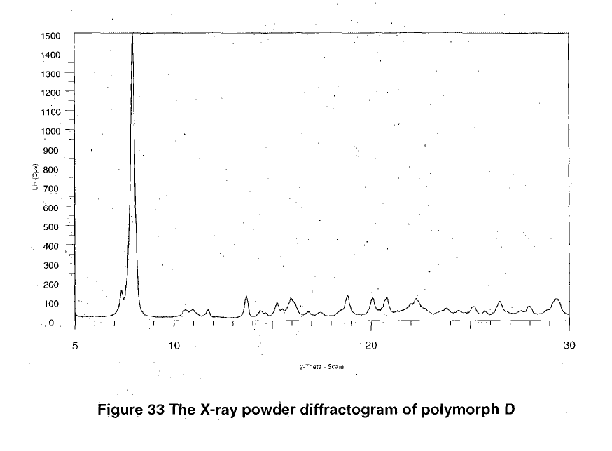

The polymorph D is characterised by having a DSC thermogram as shown in FIG. 32 and a powder X-ray diffractogram (PXRD) as shown in FIG. 33. The melting point of polymorph D from 2-butanol is 248.0° C. (onset).

### Example 14

Process for the Preparation of a Mixture of Form A and B of 6-chloro-3-(1-methylcyclopropyl)amino-4H- thieno[3,2-e]-1,2,4-thiadiazine 1,1-dioxide, Designated AB

Method 1:

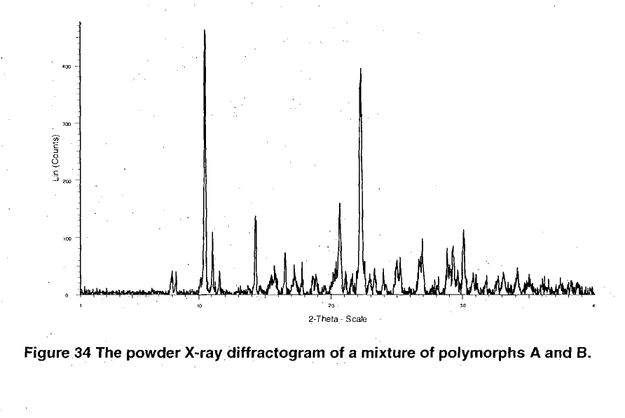

In a 500 mL reactor 15g of 6-chloro-3-(1-methylcyclopropyl)amino-4H- thieno[3,2-e]-1,2,4-thiadiazine 1,1-dioxide was suspended in 175 mL methanol and 100 mL n-butylacetate. The suspension was heated to reflux and the solution became clear. The mixture was slightly cooled and 2 g of active carbon was added. After 1 h at reflux, the solution is filtered and the solution transferred to a clean 500 mL reactor. The solution is distilled (Liebig cooler/Claisenadapter) thereby removing methanol. At a reactor temperature of 66° C. the distillation started. At 69° C., 100 mg of 6-chloro-3-(1-methylcyclopropyl)amino-4H- thieno[3,2-e]-1,2,4-thiadiazine 1,1-dioxide modification A was added and the crystallisation started. The temperature was raised to 98-99° C. Over 1 h 15 min, 250 mL of a mixture of MeOH and n-butylacetate was collected. The heating bath is removed and the mixture is allowed to cool to room temperature while stirring. The mixture was filtered. After drying to constant weight, 14 g of 6-chloro-3-(1-methylcyclopropyl)amino-4H- thieno[3,2-e]-1,2,4-thiadiazine 1,1-dioxide as a mixture of modification A and B was obtained with a powder X-ray diffractogram as shown in FIG. 34.

### Example 15

Process for the Preparation of a Mixture of form B and C of 6-chloro-3-(1-methylcyclopropyl)amino-4H- thieno[3,2-e]-1,2,4-thiadiazine 1,1-dioxide, Designated BC

Method 1:

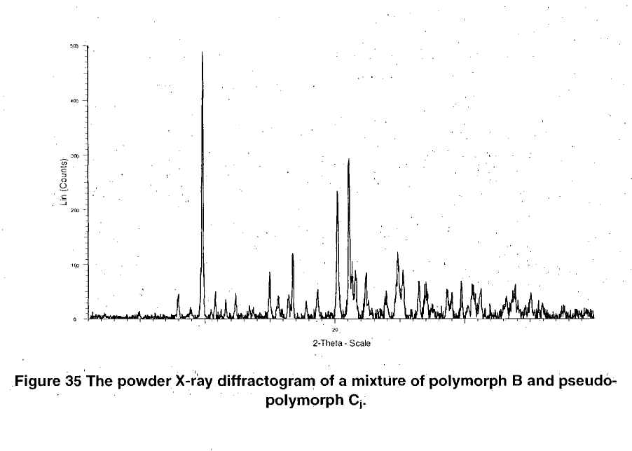

10 g of 6-chloro-3-(1-methylcyclopropyl)amino-4H- thieno[3,2-e]-1,2,4-thiadiazine 1,1-dioxide was suspended in 140 mL ethanol and 15.5 mL water. The mixture was heated to reflux. After 30 min the solution was clear. 1 g of active carbon was added and the mixture stirred for further 1 h at reflux. The mixture was filtered on a preheated filter. The filter cake was washed with a hot mixture of 9 mL ethanol and 1 mL water. To the hot filtrate (75° C.) was slowly added 100 mL water while stirring. After addition of water the mixture is slowly cooled to 5° C. and stirred for further 2 h at 5° C. to complete the crystallisation. The crystallisation started just below 60° C. The solution was filtered and the filter cake washed with 10 mL ethanol. After drying of the filter cake, 9.2 g of a mixture of form B and C was obtained with a powder X-ray diffractogram as shown in FIG. 35.

### PHARMACOLOGICAL METHODS

The ability of the compounds to interact with potassium channels can be determined by various methods. When patch-clamp techniques (Hamill O. P., Marty A., Neher E., Sakmann B. and Sigworth F. J., Plügers Arch., 391, 85-100 (1981)) are used the ionic current through a single channel of a cell can be recorded.

The activity of the compounds as potassium channel openers can also be measured as relaxation of rat aorta rings according to the following procedure:

A section of rat thoracic aorta between the aortic arch and the diaphragm. was dissected out and mounted as ring preparations as described by Taylor P. D. et al, Brit J. Pharmacol, 111, 42-48 (1994).

After a 45 min. equilibration period under a tension of 2 g, the preparations were contracted to achieve 80% of the maximum response using the required concentration of phenylephrine. When the phenylephrine response reached a plateau, potential vasodilatory agents were added cumulatively to the bath in small volumes using half log molar increments at 2 min intervals. Relaxation was expressed at the percentage of the contracted tension. The potency of a compound was expressed as the concentration required to evoke a 50% relaxation of the tissue.

In the pancreatic beta-cell the opening of the K_{ATP}-channels can be determined by measuring the subsequent change in the concentration of cytoplasmic free Ca²⁺ concentration according to the method of Arkhammar P. et al., J. Biol. Chem., 262 5448-5454 (1987).

The effect of a K_{ATP}-channel opener and a K_{ATP}-channel blocker on pancreatic beta-cells can be determined by measuring the ⁸⁶Rb⁺ efflux from a β-cell line according to the following method:

⁸⁶Rb⁺ Efflux from a β-Cell Line

The RIN 5F cell line was grown in RPMI 1640 with Glutamax I, supplemented with 10% fetal calf serum (from GibcoBRL, Scotland, UK) and maintained in an atmosphere of 5% CO₂/95% air at 37° C. The cells were detached with a Trypsin-EDTA solution (from GibcoBRL, Scotland, UK), re-suspended in medium, added 1 mCi/mL ⁸⁶Rb⁺ and replated into microtiter plates (96 well cluster 3596, sterile, from Costar Corporation, MA, USA) at a density of 50000 cells/well in 100 μu/well, and grown 24 h before use in assay.

The plates were washed 4 times with Ringer buffer (150 mM NaCl, 10 mM Hepes, 3.0 mM KCl, 1.0 mM CaCl₂, 20 mM Sucrose, pH 7.1). 80 μL Ringer buffer and 1 μL control- or test compound dissolved in DMSO was added. After incubation 1 h at room temperature with a lid, 50 μL of the supernatant was transferred to PicoPlates (Packard Instrument Company, CT, USA) and 100 μL MicroScint40 (Packard Instrument Company, CT, USA) added. The plates were counted in TopCount (Packard Instrument Company, CT, USA) for 1 min/well at the ³²P program.

The calculation of EC₅₀ and Eₘₐₓ was done by SlideWrite (Advanced Graphics Software, Inc., CA, USA) using a four parameter logistic curve: y=(a−d)/(1+(x/c)^{b})+d, where a=the activity estimated at concentration zero, b=a slope factor, c=the concentration at the middle of the curve and, d=the activity estimated at infinite concentration. EC₅₀=c and Eₘₐₓ=d, when the curve is turned of at infinite concentrations.

The effect of K_{ATP}-channel modulators on pancreatic beta-cells can be determined by measuring qualitative changes in membrane potential in the insulin producing cell line β-TC3 using fluorescence imaging techniques.

The slow fluorescent membrane potential probe DiBAC was used. The cells were kept in Ca²⁺-HEPES buffer supplemented with 10 mM glucose. After 5 sec of each 60 sec run the compound was added. 48 wells were run in each set, taking about 1 h. The same cells were then run again, now adding 25 mM KCl after 5 sec, and the depolarisation-induced increase in DiBAC fluorescence monitored for 55 sec.

In addition the effect of K_{ATP}-channel modulators on pancreatic beta-cells can be determined by measuring the increase or decrease in insulin release from insulin producing beta-cell lines or isolated islets.

Effect of K_{ATP}-channel modulators can be measured using the following procedure: The beta cells are cultured with change of media every three-four days. Cells are then seeded in 96 well microtiter dishes and cultured for three day at 38° C., 5% CO₂ and 95% humidity.

The cells are washed with NN -buffer (+10mM HEPES+0.1% BSA) for one minute and glucose (final conc. 22 mM), IBMX (final conc.0.1 mM) and compounds (final conc. from 5×10⁻⁵M−5×10⁻⁸M) added. All cells are then incubated for three h (38° C., 5% CO₂ and 95% humidity).

Supernates are harvested into Greiner minisorb microtiter wells and frozen. Insulin is measured using ELISA-techniques.

The compounds of the present invention show high potency in the insulin release test and high selectivity compared to the relaxation of rat aorta rings test.

What is claimed is:

1. A polymorphic/pseudopolymorphic form or a mixture thereof of 6-chloro-3-(1-methylcyclopropyl)amino-4H-thieno[3,2-e]-1,2,4-thiadiazine 1,1-dioxide of formula (I) 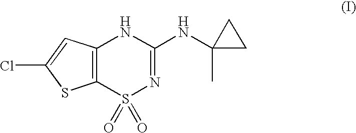
or a pharmaceutically acceptable solvate thereof.

2. A polymorphic/pseudopolymorphic form according to claim 1 obtained from the solvents acetic acid, acetone, anisole, 1-butanol, 2-butanol, butylacetate, butylmethylether, cumene, DMSO (dimethylsulfoxide), ethanol, 1-propanol, 2-propanol, ethylacetate, ethylether,ethylformate, formic acid, heptane, iso-butylacetate, iso-propylacetate, methanol, methylacetate, 3-methyl-1-butanol, methylethyl ketone, methyl iso-butyl ketone, 2-methyl-1-propanol, pentane, 1-pentanol, propylacetate, or water or any combination thereof.

3. A polymorphic form (A) according to claim 1, having the characteristics as described in Example 2.

4. A polymorphic form (B) according to claim 1, having the characteristics as described in Example 3.

5. A pseudopolymorphic form (Cj), j=1, 2, 3, 4, 5, 6, 7, 8 or 9, according to claim 1, having the characteristics as described in Examples 4, 5, 6, 7, 8, 9, 10, 11 and 12.

6. A polymorphic form (D) according to claim 1, having the characteristics as described in Example 13.

7. A mixture of polymorphic forms according to claim 1, comprising polymorphic form (A) and polymorphic form (B), having the characteristics as described in Example 14.

8. A mixture of polymorphic/pseudopolymorphic forms according to claim 1, comprising polymorphic form (B) and one of the pseudopolymorphic forms (Cj), j=1, 2, 3, 4, 5, 6, 7, 8 or 9, having the characteristic as described in Example 15.

9. A pharmaceutical composition comprising, as an active ingredient, a polymorphic/pseudopolymorphic form or a mixture thereof according to claim 1, together with one or more pharmaceutically acceptable carriers or excipients.

10. A pharmaceutical composition comprising, as an active ingredient, a polymorphic/pseudopolymorphic form or a mixture thereof according to claim 2, together with one or more pharmaceutically acceptable carriers or excipients.

11. A pharmaceutical composition comprising, as an active ingredient, the polymorphic form (A) according to claim 3, together with one or more pharmaceutically acceptable carriers or excipients.

12. A pharmaceutical composition comprising, as an active ingredient, the polymorphic form (B) according to claim 4, together with one or more pharmaceutically acceptable carriers or excipients.

13. A pharmaceutical composition comprising, as an active ingredient, the pseudopolymorphic form (Cj), j=1, 2, 3, 4, 5, 6, 7, 8 or 9, according to claim 5, together with one or more pharmaceutically acceptable carriers or excipients.

14. A pharmaceutical composition comprising, as an active ingredient, the polymorphic form (D) according to claim 6, together with one or more pharmaceutically acceptable carriers or excipients.

15. A pharmaceutical composition comprising, as an active ingredient, the mixture of polymorphic form (A) and polymorphic form (B) according to claim 7, together with one or more pharmaceutically acceptable carriers or excipients.

16. A pharmaceutical composition comprising, as an active ingredient, the mixture of polymorphic form (B) and one of the pseudopolymorphic form (Cj), j=1, 2, 3, 4, 5, 6, 7, 8 or 9, according to claim 8 together with one or more pharmaceutically acceptable carriers or excipients.

17. A pharmaceutical composition according to claim 9 in unit dosage form, comprising from about 0.05 mg to about 1000 mg, from about 0.1 to about 500 mg or from about 0.5 mg to about 200 mg per day of a polymorphic/pseudopolymorphic form or mixture thereof.

18. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the composition comprising, as an active ingredient, a polymorphic/pseudopolymorphic form or mixture thereof according to claim 1, together with one or more pharmaceutically acceptable carriers or excipients.

19. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the composition comprising, as an active ingredient,the polymorphic form (A) according to claim 3, together with one or more pharmaceutically acceptable carriers or excipients.

20. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the composition comprising, as an active ingredient, the polymorphic form (B) according to claim 4, together with one or more pharmaceutically acceptable carriers or excipients.

21. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the composition comprising, as an active ingredient, the pseudopolymorphic form (Cj), j=1, 2, 3, 4, 5, 6, 7, 8 or 9, according to claim 5, together with one or more pharmaceutically acceptable carriers or excipients.

22. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired. glucose tolerance (IGT), gestational diabetes mellitus (GDM) or obesity, the composition comprising, as an active ingredient, the polymorphic form (D) according to claim 6, together with one or more pharmaceutically acceptable carriers or excipients.

23. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the composition comprising,,as an active ingredient, the mixture of polymorphic form (A) and (B) according to claim 7, together with one or more pharmaceutically acceptable carriers or excipients.

24. A pharmaceutical composition for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the composition comprising, as an active ingredient, the mixture of polymorphic form (B) and pseudopolymorphic form (Cj), j=1, 2, 3, 4, 5, 6, 7, 8 or 9, according to claim 8, together with one or more pharmaceutically acceptable carriers or excipients.

25. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the method comprising administering to a subject in need thereof an effective amount of a polymorphic/pseudopolymorphic form or mixture thereof according to claim 1, or a pharmaceutical composition comprising the same.

26. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the method comprising administering to a subject in need thereof an effective amount of the polymorphic form (A) according to claim 3, or a pharmaceutical composition comprising the same.

27. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the method comprising administering to a subject in need thereof an effective amount of the polymorphic form (B) according to claim 4, or a pharmaceutical composition comprising the same.

28. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the method comprising administering to a subject in need thereof an effective amount of the pseudopolymorphic form (Cj), j=1, 2, 3, 4, 5, 6, 7, 8 or 9, according to claim 5, or a pharmaceutical composition comprising the same.

29. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the,method comprising administering to a subject in need thereof an effective amount of the polymorphic form (D) according to claim 6, or a pharmaceutical composition comprising the same.

30. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the method comprising administering to a subject in need thereof an effective amount of the mixture of polymorphic form (A) and (B) according to claim 7, or a pharmaceutical composition comprising the same.

31. A method for the treatment or prevention of diseases of the endocrinological system such as treatment of hyperinsulinaemia, treatment or prevention of Type-2 diabetes, prevention of Type-2 diabetes in prior GDM, prevention and intervention of Type-1 diabetes, prevention or slowing of progression of impaired fasting glucose (IFG) and impaired glucose tolerance (IGT), gestational diabetes mellitus (GDM) or obesity, the method comprising administering to a subject in need thereof an effective amount of the mixture of polymorphic form (B) and pseudopolymorphic form (Cj), j=1, 2, 3, 4, 5, 6, 7, 8 or 9, according to claim 8, or a pharmaceutical composition comprising the same.

32. A process for the preparation of polymorphic/pseudopolymorphic forms or mixtures thereof of 6-chloro-3-(1-methylcyclopropyl)amino-4H-thieno[3,2-e]-1,2,4-thiadiazine 1,1-dioxide of formula (I) 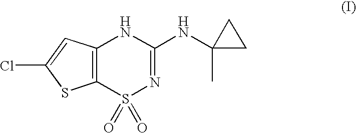
which process comprises:
a) suspending or dissolving 6-chloro-3-(1-methylcyclopropyl)amino-4H-thieno[3,2-e]-1,2,4-thiadiazine 1,1-dioxide in an appropriate solvent or a mixtures of solvents,
b) optionally heating the mixture to 60-120° C. depending on the boiling point of the appropriate solvent or solvent mixture so that the solution becomes clear, and filtering the clear solution,
c) optionally adding a co solvent at 60-120° C.,
d) optionally distilling off solvent,
e) slowly cooling the solution to 0-50° C., or adding the solution to a third solvent or mixture of solvents, or adding solvent or a mixture of solvents to the solution or combinations thereof whereby crystals are formed,
g) filtrating the resulting suspension,
h) washing the filter cake with an appropriate solvent or mixture of solvents and drying the filter cake to constant weight.
